(19) United States
(12) Patent Application Publication
(10) Pub. No.: US 2014/0005064 A1
(43) Pub. Date: Jan. 2, 2014

(54) POLYMORPHISMS IN ANGIOGENESIS PATHWAY GENES ASSOCIATED WITH TUMOR RECURRENCE IN SURGERY TREATED CANCER PATIENTS

(71) Applicant: Heinz-Josef Lenz, Altadena, CA (US)

(72) Inventor: Heinz-Josef Lenz, Altadena, CA (US)

(21) Appl. No.: 13/897,298

(22) Filed: May 17, 2013

Related U.S. Application Data
(63) Continuation-in-part of application No. 12/992,584, filed on Feb. 10, 2011.
Application No. 12/992,584 is a continuation-in-part of application No. 13/265,837, filed on Jan. 11, 2012.
(60) Provisional application No. 61/053,634, filed on May 15, 2008.
(60) Provisional application No. 61/057,758, filed on May 30, 2008.
(60) Provisional application No. 61/172,656, filed on Apr. 24, 2009.

(51) Int. Cl. C12Q 1/68

(52) U.S. Cl. CPC C12Q 1/6886
USPC 506/9; 435/6.11; 506/16

(57) ABSTRACT

This invention provides compositions and methods for determining the likely tumor recurrence of cancer patients after surgical resection. Said methods are based on determining the patient's genotype for the polymorphisms PAR-1-506 ins/del and/or EGF+61 A>G.

### CROSS REFERENCE TO RELATED APPLICATIONS

This application is a continuation-in-part of U.S. application Ser. No. 13/265,837, filed Jan. 11, 2012, which is a national stage entry under 35 U.S.C. §371 of International Application No. PCT/US2010/032319, filed Apr. 23, 2010, which in turn claims the benefit under 35 U.S.C. §119(e) of U.S. Provisional Application Nos. 61/172,656, filed Apr. 24, 2009, the contents of each of which are hereby incorporated by reference into the present disclosure. This application is also a continuation-in-part of 12/992,584, filed Feb. 10, 2011, which is a national stage entry under 35 U.S.C. §371 of International Application No. PCT/US2009/044043, filed May 14, 2009, which in turn claims the benefit under 35 U.S.C. §119(e) of U.S. Provisional Application Nos. 61/053,634, filed May 15, 2008, and 61/057,758, filed May 30, 2008, the contents of each of which are hereby incorporated by reference into the present disclosure.

### STATEMENT AS TO FEDERALLY SPONSORED RESEARCH OR DEVELOPMENT

This invention was made with government support under the National Institutes of Health Grant P30 CA 14089. Accordingly, the U.S. Government has certain rights to the invention.

### FIELD OF THE INVENTION

This invention relates to the filed of pharmacogenomics and specifically to the application of gene expression and genetic polymorphisms to diagnose and treat diseases.

### BACKGROUND OF THE INVENTION

Cancers arising from the esophagus, including the gastroesophageal junction, are becoming more common in the United States. Over the past 30 years, esophageal adenocarcinoma (EA) is the most rapidly increasing cancer in the western world and its incidence has surpassed that of esophageal squamous cell carcinoma. EA is an aggressive tumor with an overall survival (OS) rate of 15-20% (Enzinger & Mayer (2003) N Engl J Med 349(23):2241-52). Despite recent improvements in the detection (Lordick et al. (2007) Lancet Oncol 8(9):797-805), surgical resection (Holscher et al. (2007) Ann Surg 245(2):241-6; Peyre et al. (2007) Ann Surg 246(4):665-74; Hagen et al. (2001) Ann Surg 234(4):520-30; discussion 30-1) and (neo-) adjuvant radiochemotherapy (Mariette et al. (2007) Lancet Oncol 8(6):545-53), the overall survival (OS) of EA remains lower than most other solid tumors. Traditionally, surgical resection has offered the best hope for prolonged survival (Enzinger & Mayer (2003) N Engl J Med 349(23):2241-52). Transthoracic en-bloc esophagectomy with gastroplasty and two-field lymphadenectomy has offered significant improvements in local disease control and is currently considered the procedure of choice worldwide for patients with resectable middle to lower third EA (Hagen et al. (2001) Ann Surg 234(4):520-30; discussion 30-1). Nevertheless, tumor recurrence after curative resection continues to be a significant problem in the management of patients with EA. Accordingly, the development of molecular markers of prognosis as an adjunct to traditional staging systems may not only be helpful in identifying patients who are at high risk, but they will also be critical in selecting more efficient treatment strategies.

The molecular events underlying progression of Barrett's esophagus to EA remain an area of active investigation. However, neovascularization and sustained tumor angiogenesis have been studied in EAs and the so called angiogenic switch, the induction of tumor vasculature or switch to an angiogenic phenotype, is considered a hallmark of the malignant process and is required for tumor propagation and disease progression (Bergers & Benjamin (2003) Nat Rev Cancer 3(6):401-10). Although vascular endothelial growth factor (VEGF) has received considerable interest recently, the factors that regulate the switch to an angiogenic phenotype are not fully understood. Besides hypoxia driven up-regulation of VEGF expression (Stoeltzing et al. (2004) J Natl Cancer Inst 96(12):946-56), considerable evidence is accumulating supporting other mechanisms of early onset VEGF induction in vitro and in vivo (Even-Ram et al. (1998) Nat Med 4(8):909-14; Ma et al. (2005) Proc Natl Acad Sci USA 102(1):216-20); these include clinical and preclinical findings, suggesting that tumor angiogenesis is not only dependent on tumor and endothelial cells, but also on platelet-endothelium interaction. Platelets, in addition to their function in hemostasis, play an important role in wound healing and tumor growth, at least in part through the release of pro- and antiangiogenic factors; thus platelets may modulate tumor angiogenesis by releasing promoters such as VEGF and epidermal growth factor (EGF), but also endostatin (ES), a potent endogenous antiangiogenic growth factor (Italiano et al. (2008) Blood 111(3):1227-33; Pinedo et al. (1998) Lancet 352(9142):1775-7). In addition, recent evidence suggests that pro- and antiangiogenic proteins are segregated into different sets of α-granules within platelets. Interestingly, proteinase-activated receptors (PAR) have been shown to differentially counter-regulate the local release of VEGF and its antiangiogenic counter-part endostatin (ES) (Ma et al. (2005) Proc Natl Acad Sci USA 102(1):216-20; Italiano et al. (2008) Blood 111(3):1227-33). Therefore it has been proposed, that these PARs play a crucial role in the regulation of local and early-onset angiogenesis and in turn may impact the process of tumor growth and disease progression (Ma et al. (2005) Proc Natl Acad Sci USA 102(1):216-20). Based on this information, it is hypothesized that functional VEGF, VEGFR2, EGF, EGFR, PAR-1, ES, and IL-8 polymorphisms could be associated with differences in clinical outcome in patients with localized esophageal adenocarcinoma treated with surgery alone.

In nature, organisms of the same species usually differ from each other in some aspects, e.g., their appearance. The differences are genetically determined and are referred to as polymorphism. Genetic polymorphism is the occurrence in a population of two or more genetically determined alternative phenotypes due to different alleles. Polymorphism can be observed at the level of the whole individual (phenotype), in variant forms of proteins and blood group substances (biochemical polymorphism), morphological features of chromosomes (chromosomal polymorphism) or at the level of DNA in differences of nucleotides (DNA polymorphism).

Polymorphism also plays a role in determining differences in an individual's response to drugs. Pharmacogenetics and pharmacogenomics are multidisciplinary research efforts to study the relationship between genotype, gene expression profiles, and phenotype, as expressed in variability between individuals in response to or toxicity from drugs. Indeed, it is now known that cancer chemotherapy is limited by the predisposition of specific populations to drug toxicity or poor drug response. For a review of the use of germline polymorphisms in clinical oncology, see Lenz (2004) J. Clin. Oncol. 22(13):2519-2521; Park et al. (2006) Curr. Opin. Pharma. 6(4):337-344; Zhang et al. (2006) Pharma. and Genomics 16(7):475-483 and U.S. Patent Publ. No. 2006/0115827. For a review of pharmacogenetics and pharmacogenomics in therapeutic antibody development for the treatment of cancer, see Yan and Beckman (2005) Biotechniques 39:565-568.

Although considerable research correlating gene expression and/or polymorphisms has been reported, much work remains to be done. This invention supplements the existing body of knowledge and provides related advantages as well.

### BRIEF SUMMARY OF THE INVENTION

This invention provides compositions and methods for determining the likely tumor recurrence of cancer patients after surgical resection. Thus, in one aspect, this invention provides a method for identifying a patient having a cancer as more or less likely to experience tumor recurrence, or alternatively for identifying a patient having a cancer as likely or not likely to experience tumor recurrence, comprising, or alternatively consisting essentially of, or yet further consisting of, determining a genotype of a cell or tissue sample isolated from the patient for at least one polymorphism of the group PAR-1-506 ins/del or EGF+61 A>G, wherein a genotype of at least one of:

(a) (ins/ins or del/ins) for PAR-1-506 ins/del; or

(b) (A/A) for EGF+61 A>G,

identifies the patient as likely, or more likely to experience tumor recurrence, or a genotype of at least one of:

(c) (del/del) for PAR-1-506 ins/del; or

(d) (A/G or G/G) for EGF+61 A>G,

identifies the patient as not likely, or less likely to experience tumor recurrence. Alternatively, a genotype of neither (a) nor (b) identifies the patient as not likely, or less likely to experience tumor recurrence. In some embodiments, the patient is treated with surgery.

In one aspect, a genotype of at least one of:

(a) (ins/ins or del/ins) for PAR-1-506 ins/del; or

(b) (A/A) for EGF+61 A>G,

identifies the patient as likely or more likely to experience tumor recurrence.

In an alternative aspect, a genotype of at least one of:

(c) (del/del) for PAR-1-506 ins/del; or

(d) (A/G or G/G) for EGF+61 A>G,

identifies the patient as not likely or less likely to experience tumor recurrence. Alternatively, a genotype of neither (a) nor (b) identifies the patient as not likely, or less likely to experience tumor recurrence.

In some embodiments, a patient having a genotype of a group that is more likely to experience tumor recurrence is a patient that is relatively more likely to experience tumor recurrence than patients suffering from the cancer and not having a genotype of the group.

Also provided is a method for identifying a patient having a cancer treated with surgery as more or less likely to experience tumor recurrence, comprising, or alternatively consisting essentially of, or yet further consisting of, determining a genotype of a cell or tissue sample isolated from the patient for a PAR-1-506 ins/del polymorphism, wherein a genotype of (ins/ins or del/ins) for PAR-1-506 ins/del identifies the patient as more likely to experience tumor recurrence, or a genotype of (del/del) for PAR-1-506 ins/del identifies the patient as less likely to experience tumor recurrence. In one aspect, a patient more likely to experience tumor recurrence is a patient that is relatively more likely to experience tumor recurrence than patients having the cancer and having a genotype of (del/del) for PAR-1-506 ins/del.

This invention also provides a method for identifying a patient having a cancer treated with surgery as more or less likely to experience tumor recurrence, comprising, or alternatively consisting essentially of, or yet further consisting of, determining a genotype of a cell or tissue sample isolated from the patient for an EGF+61 A>G polymorphism, wherein a genotype of (A/A) for EGF+61 A>G identifies the patient as more likely to experience tumor recurrence, or a genotype of (A/G or G/G) for EGF+61 A>G identifies the patient as less likely to experience tumor recurrence. In one aspect, a patient more likely to experience tumor recurrence is a patient that is relatively more likely to experience tumor recurrence than patients having the cancer and having a genotype of (A/G or G/G) for EGF+61 A>G.

Also provided is a kit for use in determining if a cancer patient treated with surgery is more likely to experience tumor recurrence, comprising, or alternatively consisting essentially of, or yet further consisting of, suitable primers or probes for determining at least one polymorphism of the group PAR-1-506 ins/del or EGF+61 A>G, and instructions for use therein.

In one aspect of any of the above methods or kits, the cancer patient suffered at least one cancer of the type of the group: esophageal adenocarcinoma, lung cancer, breast cancer, head and neck cancer, ovarian cancer, metastatic or non-metastatic rectal cancer, metastatic or non-metastatic colon cancer, metastatic or non-metastatic colorectal cancer, or non-small cell lung cancer (NSCLC), before being treated with surgery. In one particular aspect, the cancer patient had at least esophageal adenocarcinoma before treated with surgery.

In one aspect of any of the above methods or kits, a patient that is more likely to experience tumor recurrence is a patient that has a relatively shorter time to tumor recurrence. In another aspect, a patient that is less likely to experience tumor recurrence is a patient that has a relatively longer time to tumor recurrence.

### BRIEF DESCRIPTION OF THE SEVERAL VIEWS OF THE DRAWINGS

FIG. 1a shows that the PAR-1-506 ins/del polymorphism showed a significant association with TTR. Patients with the PAR-1-506 ins/ins genotype had a median TTR of 0.9 years (95% CI: 0.5 to 3.8 years), compared to 1.9 years (95% CI: 1.1-3.1 years) and 4.0 years (95% CI: 2.7-13.2+ years) in patients heterozygous (ins/del) and homozygous (del/del) for the −506 deletion allele, respectively (p=0.003, log-rank test).

FIG. 1b shows that the EGF+61 A>G polymorphism had a significant association with TTR. patients with the EGF+61 A/A genotype had a median TTR of 1.8 years (95% CI: 0.8 to 2.8 years), compared to 3.8 years (95% CI: 2.2 to 13.2+ years) and 3.2 years (95% CI: 1.3 to 6.2+ years) for those heterozygous (A/G) and homozygous (G/G) for the +61 G-allele, respectively (p=0.034, log-rank test).

FIG. 1c shows there was a statistically significant relationship between the two polymorphisms and TTR. Patients harboring two favorable alleles were at lowest risk to develop tumor recurrence (RR=1; reference), compared to patients carrying one (RR=2.0; CI: 1.30-3.07) or no (RR=2.59; CI: 1.43-4.68) favorable alleles, who were at greater risk to develop tumor recurrence (adjusted p-value <0.001; Table 4).

### DETAILED DESCRIPTION OF THE INVENTION

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

The practice of the present invention employs, unless otherwise indicated, conventional techniques of molecular biology (including recombinant techniques), microbiology, cell biology, biochemistry and immunology, which are within the skill of the art. Such techniques are explained fully in the literature for example in the following publications. See, e.g., Sambrook and Russell eds. MOLECULAR CLONING: A LABORATORY MANUAL, 3^{rd} edition (2001); the series CURRENT PROTOCOLS 1N MOLECULAR BIOLOGY (F. M. Ausubel et al. eds. (2007)); the series METHODS IN ENZYMOLOGY (Academic Press, Inc., N.Y.); PCR 1: A PRACTICAL APPROACH (M. MacPherson et al. IRL Press at Oxford University Press (1991)); PCR 2: A PRACTICAL APPROACH (M. J. MacPherson, B. D. Hames and G. R. Taylor eds. (1995)); ANTIBODIES, A LABORATORY MANUAL (Harlow and Lane eds. (1999)); CULTURE OF ANIMAL CELLS: A MANUAL OF BASIC TECHNIQUE (R. I. Freshney 5^{th} edition (2005)); OLIGONUCLEOTIDE SYNTHESIS (M. J. Gait ed. (1984)); Mullis et al. U.S. Pat. No. 4,683,195; NUCLEIC ACID HYBRIDIZATION (B. D. Hames & S. J. Higgins eds. (1984)); NUCLEIC ACID HYBRIDIZATION (M. L. M. Anderson (1999)); TRANSCRIPTION AND TRANSLATION (B. D. Hames & S. J. Higgins eds. (1984)); IMMOBILIZED CELLS AND ENZYMES (IRL Press (1986)); B. Perbal, A PRACTICAL GUIDE TO MOLECULAR CLONING (1984); GENE TRANSFER VECTORS FOR MAMMALIAN CELLS (J. H. Miller and M. P. Calos eds. (1987) Cold Spring Harbor Laboratory); GENE TRANSFER AND EXPRESSION IN MAMMALIAN CELLS (S.C. Makrides ed. (2003)) IMMUNOCHEMICAL METHODS IN CELL AND MOLECULAR BIOLOGY (Mayer and Walker, eds., Academic Press, London (1987)); WEIR′S HANDBOOK OF EXPERIMENTAL IMMUNOLOGY (L. A. Herzenberg et al. eds (1996)).

### DEFINITIONS

As used herein, certain terms may have the following defined meanings As used in the specification and claims, the singular form “a,” “an” and “the” include singular and plural references unless the context clearly dictates otherwise. For example, the term “a cell” includes a single cell as well as a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this invention. Accordingly, it is intended that the methods and compositions can include additional steps and components (comprising) or alternatively including steps and compositions of no significance (consisting essentially of) or alternatively, intending only the stated method steps or compositions (consisting of).

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. The term “about” also includes the exact value “X” in addition to minor increments of “X” such as “X+0.1” or “X−0.1.” It also is to be understood, although not always explicitly stated, that the reagents described herein are merely exemplary and that equivalents of such are known in the art.

The term “identify” or “identifying” is to associate or affiliate a patient closely to a group or population of patients who likely experience the same or a similar clinical response to treatment.

A “normal cell corresponding to the tumor tissue type” refers to a normal cell from a same tissue type as the tumor tissue. A non-limiting examples is a normal lung cell from a patient having lung tumor, or a normal colon cell from a patient having colon tumor.

A “blood cell” refers to any of the cells contained in blood. A blood cell is also referred to as an erythrocyte or leukocyte, or a blood corpuscle. Non-limiting examples of blood cells include white blood cells, red blood cells, and platelets.

The term “adjuvant” therapy refers to administration of a therapy or chemotherapeutic regimen to a patient after removal of a tumor by surgery. Adjuvant therapy is typically given to minimize or prevent a possible cancer reoccurrence. Alternatively, “neoadjuvant” therapy refers to administration of therapy or chemotherapeutic regimen before surgery, typically in an attempt to shrink the tumor prior to a surgical procedure to minimize the extent of tissue removed during the procedure.

The term “allele,” which is used interchangeably herein with “allelic variant” refers to alternative forms of a gene or portions thereof. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene. Alleles of a specific gene can differ from each other in a single nucleotide, or several nucleotides, and can include substitutions, deletions and insertions of nucleotides. An allele of a gene can also be a form of a gene containing a mutation.

As used herein, the term “determining the genotype of a cell or tissue sample” intends to identify the genotypes of polymorphic loci of interest in the cell or tissue sample. In one aspect, a polymorphic locus is a single nucleotide polymorphic (SNP) locus. If the allelic composition of a SNP locus is heterozygous, the genotype of the SNP locus will be identified as “X/Y” wherein X and Y are two different nucleotides, e.g., A/G for the EGF gene at position +61. If the allelic composition of a SNP locus is heterozygous, the genotype of the SNP locus will be identified as “X/X” wherein X identifies the nucleotide that is present at both alleles, e.g., G/G for the EGF gene at position +61. In another aspect, a polymorphic locus harbors allelic variants of nucleotide sequences of different length. The genotype of the polymorphic locus will be identified with the length of the allelic variant, e.g., both alleles with <20 CA repeats at intron 1 of the EGFR gene. The genotype of the cell or tissue sample will be identified as a combination of genotypes of all polymorphic loci of interest, e.g. A/G for the EGF gene at position +61 and both alleles with <20 CA repeats at intron 1 of the EGFR gene.

The term “genetic marker” refers to an allelic variant of a polymorphic region of a gene of interest and/or the expression level of a gene of interest.

The term “wild-type allele” refers to an allele of a gene which, when present in two copies in a subject results in a wild-type phenotype. There can be several different wild-type alleles of a specific gene, since certain nucleotide changes in a gene may not affect the phenotype of a subject having two copies of the gene with the nucleotide changes.

The term “polymorphism” refers to the coexistence of more than one form of a gene or portion thereof. A portion of a gene of which there are at least two different forms, i.e., two different nucleotide sequences, is referred to as a “polymorphic region of a gene.” A polymorphic region can be a single nucleotide, the identity of which differs in different alleles.

A “polymorphic gene” refers to a gene having at least one polymorphic region.

The term “genotype” refers to the specific allelic composition of an entire cell or a certain gene and in some aspects a specific polymorphism associated with that gene, whereas the term “phenotype” refers to the detectable outward manifestations of a specific genotype.

The phrase “amplification of polynucleotides” includes methods such as PCR, ligation amplification (or ligase chain reaction, LCR) and amplification methods. These methods are known and widely practiced in the art. See, e.g., U.S. Pat. Nos. 4,683,195 and 4,683,202 and Innis et al., 1990 (for PCR); and Wu, D. Y. et al. (1989) Genomics 4:560-569 (for LCR). In general, the PCR procedure describes a method of gene amplification which is comprised of (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e. each primer is specifically designed to be complementary to each strand of the genomic locus to be amplified.

Reagents and hardware for conducting PCR are commercially available. Primers useful to amplify sequences from a particular gene region are preferably complementary to, and hybridize specifically to sequences in the target region or in its flanking regions. Nucleic acid sequences generated by amplification may be sequenced directly. Alternatively the amplified sequence(s) may be cloned prior to sequence analysis. A method for the direct cloning and sequence analysis of enzymatically amplified genomic segments is known in the art.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

The term “interact” as used herein is meant to include detectable interactions between molecules, such as can be detected using, for example, a hybridization assay. The term interact is also meant to include “binding” interactions between molecules. Interactions may be, for example, protein-protein, protein-nucleic acid, protein-small molecule or small molecule-nucleic acid in nature.

The term “isolated” as used herein refers to molecules or biological or cellular materials being substantially free from other materials. In one aspect, the term “isolated” refers to nucleic acid, such as DNA or RNA, or protein or polypeptide, or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

When a genetic marker or polymorphism “is used as a basis” for identifying or selecting a patient for a treatment described herein, the genetic marker or polymorphism is measured before and/or during treatment, and the values obtained are used by a clinician in assessing any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); (e) probable or likely unsuitability of an individual to continue to receive treatment(s); (f) adjusting dosage; (g) predicting likelihood of clinical benefits; or (h) toxicity. As would be well understood by one in the art, measurement of the genetic marker or polymorphism in a clinical setting is a clear indication that this parameter was used as a basis for initiating, continuing, adjusting and/or ceasing administration of the treatments described herein.

As used herein, the term “patient” intends an animal, a mammal or yet further a human patient. For the purpose of illustration only, a mammal includes but is not limited to a human, a simian, a murine, a bovine, an equine, a porcine or an ovine.

The term “treating” as used herein is intended to encompass curing as well as ameliorating at least one symptom of the condition or disease. For example, in the case of cancer, a response to treatment includes a reduction in cachexia, increase in survival time, elongation in time to tumor progression, reduction in tumor mass, reduction in tumor burden and/or a prolongation in time to tumor metastasis, time to tumor recurrence, tumor response, complete response, partial response, stable disease, progressive disease, progression free survival, overall survival, each as measured by standards set by the National Cancer Institute and the U.S. Food and Drug Administration for the approval of new drugs. See Johnson et al. (2003) J. Clin. Oncol. 21(7):1404-1411.

“An effective amount” intends to indicated the amount of a compound or agent administered or delivered to the patient which is most likely to result in the desired response to treatment. The amount is empirically determined by the patient's clinical parameters including, but not limited to the Stage of disease, age, gender, histology, and likelihood for tumor recurrence.

The term “clinical outcome”, “clinical parameter”, “clinical response”, or “clinical endpoint” refers to any clinical observation or measurement relating to a patient's reaction to a therapy. Non-limiting examples of clinical outcomes include tumor response (TR), overall survival (OS), progression free survival (PFS), disease free survival, time to tumor recurrence (TTR), time to tumor progression (TTP), relative risk (RR), toxicity or side effect.

The term “likely to respond” intends to mean that the patient of a genotype is relatively more likely to experience a complete response or partial response than patients similarly situated without the genotype. Alternatively, the term “not likely to respond” intends to mean that the patient of a genotype is relatively less likely to experience a complete response or partial response than patients similarly situated without the genotype.

The term “suitable for a therapy” or “suitably treated with a therapy” shall mean that the patient is likely to exhibit one or more desirable clinical outcome as compared to patients having the same disease and receiving the same therapy but possessing a different characteristic that is under consideration for the purpose of the comparison. In one aspect, the characteristic under consideration is a genetic polymorphism or a somatic mutation. In another aspect, the characteristic under consideration is expression level of a gene or a polypeptide. In one aspect, a more desirable clinical outcome is relatively higher likelihood of or relatively better tumor response such as tumor load reduction. In another aspect, a more desirable clinical outcome is relatively longer overall survival. In yet another aspect, a more desirable clinical outcome is relatively longer progression free survival or time to tumor progression. In yet another aspect, a more desirable clinical outcome is relatively longer disease free survival. In further another aspect, a more desirable clinical outcome is relative reduction or delay in tumor recurrence. In another aspect, a more desirable clinical outcome is relatively decreased metastasis. In another aspect, a more desirable clinical outcome is relatively lower relative risk. In yet another aspect, a more desirable clinical outcome is relatively reduced toxicity or side effects. In some embodiments, more than one clinical outcomes are considered simultaneously. In one such aspect, a patient possessing a characteristic, such as a genotype of a genetic polymorphism, may exhibit more than one more desirable clinical outcomes as compared to patients having the same disease and receiving the same therapy but not possessing the characteristic. As defined herein, the patients is considered suitable for the therapy. In another such aspect, a patient possessing a characteristic may exhibit one or more desirable clinical outcome but simultaneously exhibit one or more less desirable clinical outcome. The clinical outcomes will then be considered collectively, and a decision as to whether the patient is suitable for the therapy will be made accordingly, taking into account the patient's specific situation and the relevance of the clinical outcomes. In some embodiments, progression free survival or overall survival is weighted more heavily than tumor response in a collective decision making.

A “complete response” (CR) to a therapy defines patients with evaluable but non-measurable disease, whose tumor and all evidence of disease had disappeared.

A “partial response” (PR) to a therapy defines patients with anything less than complete response that were simply categorized as demonstrating partial response.

“Stable disease” (SD) indicates that the patient is stable.

“Progressive disease” (PD) indicates that the tumor has grown (i.e. become larger), spread (i.e. metastasized to another tissue or organ) or the overall cancer has gotten worse following treatment. For example, tumor growth of more than 20 percent since the start of treatment typically indicates progressive disease. “Disease free survival” indicates the length of time after treatment of a cancer or tumor during which a patient survives with no signs of the cancer or tumor.

“Non-response” (NR) to a therapy defines patients whose tumor or evidence of disease has remained constant or has progressed.

“Overall Survival” (OS) intends a prolongation in life expectancy as compared to naïve or untreated individuals or patients.

“Progression free survival” (PFS) or “Time to Tumor Progression” (TTP) indicates the length of time during and after treatment that the cancer does not grow. Progression-free survival includes the amount of time patients have experienced a complete response or a partial response, as well as the amount of time patients have experienced stable disease.

“No Correlation” refers to a statistical analysis showing no relationship between the allelic variant of a polymorphic region or gene expression levels and clinical parameters.

“Tumor Recurrence” as used herein and as defined by the National Cancer Institute is cancer that has recurred (come back), usually after a period of time during which the cancer could not be detected. The cancer may come back to the same place as the original (primary) tumor or to another place in the body. It is also called recurrent cancer.

“Time to Tumor Recurrence” (TTR) is defined as the time from the date of diagnosis of the cancer to the date of first recurrence, death, or until last contact if the patient was free of any tumor recurrence at the time of last contact. If a patient had not recurred, then TTR was censored at the time of death or at the last follow-up.

“Relative Risk” (RR), in statistics and mathematical epidemiology, refers to the risk of an event (or of developing a disease) relative to exposure. Relative risk is a ratio of the probability of the event occurring in the exposed group versus a non-exposed group.

As used herein, the terms “Stage I cancer,” “Stage II cancer,” “Stage III cancer,” and “Stage IV” refer to the TNM staging classification for cancer. Stage I cancer typically identifies that the primary tumor is limited to the organ of origin. Stage II intends that the primary tumor has spread into surrounding tissue and lymph nodes immediately draining the area of the tumor. Stage III intends that the primary tumor is large, with fixation to deeper structures. Stage IV intends that the primary tumor is large, with fixation to deeper structures. See pages 20 and 21, CANCER BIOLOGY, 2^{nd} Ed., Oxford University Press (1987).

A “tumor” is an abnormal growth of tissue resulting from uncontrolled, progressive multiplication of cells and serving no physiological function. A “tumor” is also known as a neoplasm.

“Having the same cancer” is used when comparing one patient to another or alternatively, one patient population to another patient population. For example, the two patients or patient population will each have or be suffering from colon cancer.

The term “blood” refers to blood which includes all components of blood circulating in a subject including, but not limited to, red blood cells, white blood cells, plasma, clotting factors, small proteins, platelets and/or cryoprecipitate. This is typically the type of blood which is donated when a human patent gives blood.

### DESCRIPTIVE EMBODIMENTS

The invention further provides diagnostic and prognostic methods, which are based, at least in part, on determination of a genetic polymorphism in a gene of interest identified herein.

For example, information obtained using the diagnostic assays described herein is useful for determining if a subject is likely to experience tumor recurrence. Based on the prognostic information, a doctor can recommend a follow-up therapeutic protocol, useful for reducing the malignant mass or tumor in the patient or treat cancer in the individual.

It is to be understood that information obtained using the diagnostic assays described herein may be used alone or in combination with other information, such as, but not limited to, genotypes or expression levels of other genes, clinical chemical parameters, histopathological parameters, or age, gender and weight of the subject. When used alone, the information obtained using the diagnostic assays described herein is useful in determining or identifying the clinical outcome of a treatment, likely side effects, selecting a patient for a treatment, or treating a patient, etc. When used in combination with other information, on the other hand, the information obtained using the diagnostic assays described herein is useful in aiding in the determination or identification of clinical outcome of a treatment, aiding in the selection of a patient for a treatment, or aiding in the treatment of a patient and etc. In a particular aspect, the identify of a polymorphism at a position within a gene of interest are used in a panel of genes, each of which contributes to the final diagnosis, prognosis or treatment.

The methods are useful in the assistance of an animal, a mammal or yet further a human patient. For the purpose of illustration only, a mammal includes but is not limited to a human patient, a simian, a murine, a bovine, an equine, a porcine or an ovine.

Provided, in one aspect, is a method for identifying a patient having a cancer as more or less likely to experience tumor recurrence, or alternatively for identifying a patient having a cancer as likely or not likely to experience tumor recurrence, comprising, or alternatively consisting essentially of, or yet further consisting of, determining a genotype of a cell or tissue sample isolated from the patient for at least one polymorphism of the group PAR-1-506 ins/del or EGF+61 A>G, wherein a genotype of at least one of:

(a) (ins/ins or del/ins) for PAR-1-506 ins/del; or

(b) (A/A) for EGF+61 A>G,

identifies the patient as likely, or more likely to experience tumor recurrence, or a genotype of at least one of:

(c) (del/del) for PAR-1-506 ins/del; or

(d) (A/G or G/G) for EGF+61 A>G,

identifies the patient as not likely, or less likely to experience tumor recurrence. Alternatively, a genotype of neither (a) nor (b) identifies the patient as not likely, or less likely to experience tumor recurrence. In some embodiments, the patient is treated with surgery.

In one aspect, a genotype of at least one of:

(a) (ins/ins or del/ins) for PAR-1-506 ins/del; or

(b) (A/A) for EGF+61 A>G,

identifies the patient as likely or more likely to experience tumor recurrence.

In another aspect, a genotype of at least one of:

(c) (del/del) for PAR-1-506 ins/del; or

(d) (A/G or G/G) for EGF+61 A>G,

identifies the patient as not likely or less likely to experience tumor recurrence. Alternatively, a genotype of neither (a) nor (b) identifies the patient as not likely, or less likely to experience tumor recurrence.

In some embodiments, a patient having a genotype of a group that is more likely to experience tumor recurrence is a patient that is relatively more likely to experience tumor recurrence than patients suffering from the cancer and not having a genotype of the group.

Also provided is a method for identifying a patient having a cancer treated with surgery as more or less likely to experience tumor recurrence, comprising, or alternatively consisting essentially of, or yet further consisting of, determining a genotype of a cell or tissue sample isolated from the patient for a PAR-1-506 ins/del polymorphism, wherein a genotype of (ins/ins or del/ins) for PAR-1-506 ins/del identifies the patient as more likely to experience tumor recurrence, or a genotype of (del/del) for PAR-1-506 ins/del identifies the patient as less likely to experience tumor recurrence. In one aspect, a patient more likely to experience tumor recurrence is a patient that is relatively more likely to experience tumor recurrence than patients having the cancer and having a genotype of (del/del) for PAR-1-506 ins/del.

This invention further provides a method for identifying a patient having a cancer treated with surgery as more or less likely to experience tumor recurrence, comprising, or alternatively consisting essentially of, or yet further consisting of, determining a genotype of a cell or tissue sample isolated from the patient for an EGF+61 A>G polymorphism, wherein a genotype of (A/A) for EGF+61 A>G identifies the patient as more likely to experience tumor recurrence, or a genotype of (A/G or G/G) for EGF+61 A>G identifies the patient as less likely to experience tumor recurrence. In one aspect, a patient more likely to experience tumor recurrence is a patient that is relatively more likely to experience tumor recurrence than patients having the cancer and having a genotype of (A/G or G/G) for EGF+61 A>G.

In one aspect of any of the above methods, the cancer patient suffered at least one cancer of the type of the group: esophageal adenocarcinoma, lung cancer, breast cancer, head and neck cancer, ovarian cancer, metastatic or non-metastatic rectal cancer, metastatic or non-metastatic colon cancer, metastatic or non-metastatic colorectal cancer, or non-small cell lung cancer (NSCLC), before being treated with surgery. In a particular aspect, the cancer patient had at least esophageal adenocarcinoma before being treated with surgery.

In one aspect of the above noted methods, the cancer patient, after surgical resection, underwent chemotherapy or radiotherapy. Non-limiting examples of chemotherapy include administration of anti-VEGF antibodies such as Bevacizumab, anti-EGFR antibodies such as Cetuximab, topoisomerase inhibitors such as Irinotecan (CTP-11), platinum drugs such as carboplatin or oxaliplatin, mitotic inhibitors such as paclitaxel, pyrimidine antimetabolites such as 5-FU or capecitabin, or FOLFOX or XELOX.

In some embodiments, a patient that is more likely to experience tumor recurrence is a patient that has a relatively shorter time to tumor recurrence. In an alternative aspect, a patient that is less likely to experience tumor recurrence is a patient that has a relatively longer time to tumor recurrence.

In some embodiments of the above noted methods, the sample is at least one of a fixed tissue, a frozen tissue, a biopsy tissue, a resection tissue, a microdissected tissue, or combinations thereof.

Suitable patient samples in the methods include, but are not limited to a sample comprises, or alternatively consisting essentially of, or yet further consisting of, at least one of a tumor cell, a normal cell adjacent to a tumor, a normal cell corresponding to the tumor tissue type, a blood cell, a peripheral blood lymphocyte, or combinations thereof. The samples can be at least one of a fixed tissue, a frozen tissue, a biopsy tissue, a resection tissue, a microdissected tissue, or combinations thereof.

Any suitable method for identifying the genotype in the patient sample can be used and the inventions described herein are not to be limited to these methods. For the purpose of illustration only, the genotype is determined by a method comprising, or alternatively consisting essentially of, or yet further consisting of, hybridization, PCR or more specifically, PCR-RFLP or microarray. These methods as well as equivalents or alternatives thereto are described herein.

The methods are useful in the assistance of an animal, a mammal or yet further a human patient. For the purpose of illustration only, a mammal includes but is not limited to a simian, a murine, a bovine, an equine, a porcine or an ovine.

### Diagnostic Methods

The invention further provides diagnostic, prognostic and therapeutic methods, which are based, at least in part, on determination of the identity of the polymorphic region of the genes identified herein.

### Polymorphic Region

For example, information obtained using the diagnostic assays described herein is useful for determining if a subject will likely, more likely, or less likely to respond to cancer treatment of a given type. Based on the prognostic information, a doctor can recommend a therapeutic protocol, useful for treating reducing the malignant mass or tumor in the patient or treat cancer in the individual.

In addition, knowledge of the identity of a particular allele in an individual (the gene profile) allows customization of therapy for a particular disease to the individual's genetic profile, the goal of “pharmacogenomics”. For example, an individual's genetic profile can enable a doctor: 1) to more effectively prescribe a drug that will address the molecular basis of the disease or condition; 2) to better determine the appropriate dosage of a particular drug and 3) to identify novel targets for drug development. The identity of the genotype or expression patterns of individual patients can then be compared to the genotype or expression profile of the disease to determine the appropriate drug and dose to administer to the patient.

The ability to target populations expected to show the highest clinical benefit, based on the normal or disease genetic profile, can enable: 1) the repositioning of marketed drugs with disappointing market results; 2) the rescue of drug candidates whose clinical development has been discontinued as a result of safety or efficacy limitations, which are patient subgroup-specific; and 3) an accelerated and less costly development for drug candidates and more optimal drug labeling.

Detection of point mutations or additional base pair repeats can be accomplished by molecular cloning of the specified allele and subsequent sequencing of that allele using techniques known in the art, in some aspects, after isolation of a suitable nucleic acid sample using methods known in the art. Alternatively, the gene sequences can be amplified directly from a genomic DNA preparation from the tumor tissue using PCR, and the sequence composition is determined from the amplified product. As described more fully below, numerous methods are available for isolating and analyzing a subject's DNA for mutations at a given genetic locus such as the gene of interest.

A detection method is allele specific hybridization using probes overlapping the polymorphic site and having about 5, or alternatively 10, or alternatively 20, or alternatively 25, or alternatively 30 nucleotides around the polymorphic region. In another embodiment of the invention, several probes capable of hybridizing specifically to the allelic variant are attached to a solid phase support, e.g., a “chip”. Oligonucleotides can be bound to a solid support by a variety of processes, including lithography. For example a chip can hold up to 250,000 oligonucleotides (GeneChip, Affymetrix). Mutation detection analysis using these chips comprising oligonucleotides, also termed “DNA probe arrays” is described e.g., in Cronin et al. (1996) Human Mutation 7:244.

In other detection methods, it is necessary to first amplify at least a portion of the gene of interest prior to identifying the allelic variant. Amplification can be performed, e.g., by PCR and/or LCR, according to methods known in the art. In one embodiment, genomic DNA of a cell is exposed to two PCR primers and amplification for a number of cycles sufficient to produce the required amount of amplified DNA.

Alternative amplification methods include: self sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), or any other nucleic acid amplification method, followed by the detection of the amplified molecules using techniques known to those of skill in the art. These detection schemes are useful for the detection of nucleic acid molecules if such molecules are present in very low numbers.

In one embodiment, any of a variety of sequencing reactions known in the art can be used to directly sequence at least a portion of the gene of interest and detect allelic variants, e.g., mutations, by comparing the sequence of the sample sequence with the corresponding wild-type (control) sequence. Exemplary sequencing reactions include those based on techniques developed by Maxam and Gilbert (1997) Proc. Natl. Acad. Sci, USA 74:560) or Sanger et al. (1977) Proc. Nat. Acad. Sci, 74:5463). It is also contemplated that any of a variety of automated sequencing procedures can be utilized when performing the subject assays (Biotechniques (1995) 19:448), including sequencing by mass spectrometry (see, for example, U.S. Pat. No. 5,547,835 and International Patent Application Publication Number WO 94/16101, entitled DNA Sequencing by Mass Spectrometry by Koster; U.S. Pat. No. 5,547,835 and international patent application Publication Number WO 94/21822 entitled “DNA Sequencing by Mass Spectrometry Via Exonuclease Degradation” by Koster; U.S. Pat. No. 5,605,798 and International Patent Application No. PCT/US96/03651 entitled DNA Diagnostics Based on Mass Spectrometry by Koster; Cohen et al. (1996) Adv. Chromat. 36:127-162; and Griffin et al. (1993) Appl. Biochem. Bio. 38:147-159). It will be evident to one skilled in the art that, for certain embodiments, the occurrence of only one, two or three of the nucleic acid bases need be determined in the sequencing reaction. For instance, A-track or the like, e.g., where only one nucleotide is detected, can be carried out.

Yet other sequencing methods are disclosed, e.g., in U.S. Pat. No. 5,580,732 entitled “Method of DNA Sequencing Employing A Mixed DNA-Polymer Chain Probe” and U.S. Pat. No. 5,571,676 entitled “Method For Mismatch-Directed In Vitro DNA Sequencing.”

In some cases, the presence of the specific allele in DNA from a subject can be shown by restriction enzyme analysis. For example, the specific nucleotide polymorphism can result in a nucleotide sequence comprising a restriction site which is absent from the nucleotide sequence of another allelic variant.

In a further embodiment, protection from cleavage agents (such as a nuclease, hydroxylamine or osmium tetroxide and with piperidine) can be used to detect mismatched bases in RNA/RNA DNA/DNA, or RNA/DNA heteroduplexes (see, e.g., Myers et al. (1985) Science 230:1242). In general, the technique of “mismatch cleavage” starts by providing heteroduplexes formed by hybridizing a control nucleic acid, which is optionally labeled, e.g., RNA or DNA, comprising a nucleotide sequence of the allelic variant of the gene of interest with a sample nucleic acid, e.g., RNA or DNA, obtained from a tissue sample. The double-stranded duplexes are treated with an agent which cleaves single-stranded regions of the duplex such as duplexes formed based on basepair mismatches between the control and sample strands. For instance, RNA/DNA duplexes can be treated with RNase and DNA/DNA hybrids treated with 51 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine whether the control and sample nucleic acids have an identical nucleotide sequence or in which nucleotides they are different. See, for example, U.S. Pat. No. 6,455,249, Cotton et al. (1988) Proc. Natl. Acad. Sci. USA 85:4397; Saleeba et al. (1992) Methods Enzy. 217:286-295. In another embodiment, the control or sample nucleic acid is labeled for detection.

In other embodiments, alterations in electrophoretic mobility is used to identify the particular allelic variant. For example, single strand conformation polymorphism (SSCP) may be used to detect differences in electrophoretic mobility between mutant and wild type nucleic acids (Orita et al. (1989) Proc. Natl. Acad. Sci. USA 86:2766; Cotton (1993) Mutat. Res. 285:125-144 and Hayashi (1992) Genet Anal Tech. Appl. 9:73-79). Single-stranded DNA fragments of sample and control nucleic acids are denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In another preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment, the identity of the allelic variant is obtained by analyzing the movement of a nucleic acid comprising the polymorphic region in polyacrylamide gels containing a gradient of denaturant, which is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing agent gradient to identify differences in the mobility of control and sample DNA (Rosenbaum and Reissner (1987) Biophys. Chem. 265:1275).

Examples of techniques for detecting differences of at least one nucleotide between 2 nucleic acids include, but are not limited to, selective oligonucleotide hybridization, selective amplification, or selective primer extension. For example, oligonucleotide probes may be prepared in which the known polymorphic nucleotide is placed centrally (allele-specific probes) and then hybridized to target DNA under conditions which permit hybridization only if a perfect match is found (Saiki et al. (1986) Nature 324:163); Saiki et al. (1989) Proc. Natl. Acad. Sci. USA 86:6230 and Wallace et al. (1979) Nucl. Acids Res. 6:3543). Such allele specific oligonucleotide hybridization techniques may be used for the detection of the nucleotide changes in the polymorphic region of the gene of interest. For example, oligonucleotides having the nucleotide sequence of the specific allelic variant are attached to a hybridizing membrane and this membrane is then hybridized with labeled sample nucleic acid. Analysis of the hybridization signal will then reveal the identity of the nucleotides of the sample nucleic acid.

Alternatively, allele specific amplification technology which depends on selective PCR amplification may be used in conjunction with the instant invention. Oligonucleotides used as primers for specific amplification may carry the allelic variant of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238 and Newton et al. (1989) Nucl. Acids Res. 17:2503). This technique is also termed “PROBE” for Probe Oligo Base Extension. In addition it may be desirable to introduce a novel restriction site in the region of the mutation to create cleavage-based detection (Gasparini et al. (1992) Mol. Cell. Probes 6:1).

In another embodiment, identification of the allelic variant is carried out using an oligonucleotide ligation assay (OLA), as described, e.g., in U.S. Pat. No. 4,998,617 and in Landegren et al. (1988) Science 241:1077-1080. The OLA protocol uses two oligonucleotides which are designed to be capable of hybridizing to abutting sequences of a single strand of a target. One of the oligonucleotides is linked to a separation marker, e.g., biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson et al. have described a nucleic acid detection assay that combines attributes of PCR and OLA (Nickerson et al. (1990) Proc. Natl. Acad. Sci. (U.S.A.) 87:8923-8927). In this method, PCR is used to achieve the exponential amplification of target DNA, which is then detected using OLA.

Several techniques based on this OLA method have been developed and can be used to detect the specific allelic variant of the polymorphic region of the gene of interest. For example, U.S. Pat. No. 5,593,826 discloses an OLA using an oligonucleotide having 3′-amino group and a 5′-phosphorylated oligonucleotide to form a conjugate having a phosphoramidate linkage. In another variation of OLA described in Tobe et al. (1996) Nucleic Acids Res. 24: 3728, OLA combined with PCR permits typing of two alleles in a single microtiter well. By marking each of the allele-specific primers with a unique hapten, i.e. digoxigenin and fluorescein, each OLA reaction can be detected by using hapten specific antibodies that are labeled with different enzyme reporters, alkaline phosphatase or horseradish peroxidase. This system permits the detection of the two alleles using a high throughput format that leads to the production of two different colors.

In one embodiment, the single base polymorphism can be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In another embodiment of the invention, a solution-based method is used for determining the identity of the nucleotide of the polymorphic site. Cohen, D. et al. (French Patent 2,650,840; PCT Appln. No. WO91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA™ is described by Goelet, P. et al. (PCT Appln. No. 92/15712). This method uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Appln. No. WO91/02087) the method of Goelet, P. et al. supra, is preferably a heterogeneous phase assay, in which the primer or the target molecule is immobilized to a solid phase.

Several primer-guided nucleotide incorporation procedures for assaying polymorphic sites in DNA have been described (Komher, J. S. et al. (1989) Nucl. Acids. Res. 17:7779-7784; Sokolov, B. P. (1990) Nucl. Acids Res. 18:3671; Syvanen, A.-C. et al. (1990) Genomics 8:684-692; Kuppuswamy, M. N. et al. (1991) Proc. Natl. Acad. Sci. (U.S.A.) 88:1143-1147; Prezant, T. R. et al. (1992) Hum. Mutat. 1:159-164; Ugozzoli, L. et al. (1992) GATA 9:107-112; Nyren, P. et al. (1993) Anal. Biochem. 208:171-175). These methods differ from GBA™ in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A.-C. et al. (1993) Amer. J. Hum. Genet. 52:46-59).

If the polymorphic region is located in the coding region of the gene of interest, yet other methods than those described above can be used for determining the identity of the allelic variant. For example, identification of the allelic variant, which encodes a mutated signal peptide, can be performed by using an antibody specifically recognizing the mutant protein in, e.g., immunohistochemistry or immunoprecipitation. Antibodies to the wild-type or signal peptide mutated forms of the signal peptide proteins can be prepared according to methods known in the art.

Often a solid phase support is used as a support capable of binding of a primer, probe, polynucleotide, an antigen or an antibody. Well-known supports include glass, polystyrene, polypropylene, polyethylene, dextran, nylon, amylases, natural and modified celluloses, polyacrylamides, gabbros, and magnetite. The nature of the support can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. or alternatively polystyrene beads. Those skilled in the art will know many other suitable supports for binding antibody or antigen, or will be able to ascertain the same by use of routine experimentation.

Moreover, it will be understood that any of the above methods for detecting alterations in a gene or gene product or polymorphic variants can be used to monitor the course of treatment or therapy.

The methods described herein may be performed, for example, by utilizing pre-packaged diagnostic kits, such as those described below, comprising at least one probe or primer nucleic acid described herein, which may be conveniently used, e.g., to determine whether a subject is likely to experience tumor recurrence following therapy as described herein or has or is at risk of developing disease such as colon cancer.

Sample nucleic acid for use in the above-described diagnostic and prognostic methods can be obtained from any suitable cell type or tissue of a subject. For example, a subject's bodily fluid (e.g. blood) can be obtained by known techniques (e.g., venipuncture). Alternatively, nucleic acid tests can be performed on dry samples (e.g., hair or skin). Diagnostic procedures can also be performed in situ directly upon tissue sections (fixed and/or frozen) of patient tissue obtained from biopsies or resections, such that no nucleic acid purification is necessary. Nucleic acid reagents can be used as probes and/or primers for such in situ procedures (see, for example, Nuovo, G. J. (1992) PCR 1N SITU HYBRIDIZATION: PROTOCOLS AND APPLICATIONS, Raven Press, NY).

In addition to methods which focus primarily on the detection of one nucleic acid sequence, profiles can also be assessed in such detection schemes. Fingerprint profiles can be generated, for example, by utilizing a differential display procedure, Northern analysis and/or RT-PCR.

Antibodies directed against wild type or mutant peptides encoded by the allelic variants of the gene of interest may also be used in disease diagnostics and prognostics. Such diagnostic methods, may be used to detect abnormalities in the level of expression of the peptide, or abnormalities in the structure and/or tissue, cellular, or subcellular location of the peptide. Protein from the tissue or cell type to be analyzed may easily be detected or isolated using techniques which are well known to one of skill in the art, including but not limited to Western blot analysis. For a detailed explanation of methods for carrying out Western blot analysis, see Sambrook and Russell (2001) supra. The protein detection and isolation methods employed herein can also be such as those described in Harlow and Lane, (1999) supra. This can be accomplished, for example, by immunofluorescence techniques employing a fluorescently labeled antibody (see below) coupled with light microscopic, flow cytometric, or fluorimetric detection. The antibodies (or fragments thereof) useful in the present invention may, additionally, be employed histologically, as in immunofluorescence or immunoelectron microscopy, for in situ detection of the peptides or their allelic variants. In situ detection may be accomplished by removing a histological specimen from a patient, and applying thereto a labeled antibody of the present invention. The antibody (or fragment) is preferably applied by overlaying the labeled antibody (or fragment) onto a biological sample. Through the use of such a procedure, it is possible to determine not only the presence of the subject polypeptide, but also its distribution in the examined tissue. Using the present invention, one of ordinary skill will readily perceive that any of a wide variety of histological methods (such as staining procedures) can be modified in order to achieve such in situ detection.

In one embodiment, it is necessary to first amplify at least a portion of the gene of interest prior to identifying the polymorphic region of the gene of interest in a sample. Amplification can be performed, e.g., by PCR and/or LCR, according to methods known in the art. Various non-limiting examples of PCR include the herein described methods.

Allele-specific PCR is a diagnostic or cloning technique is used to identify or utilize single-nucleotide polymorphisms (SNPs). It requires prior knowledge of a DNA sequence, including differences between alleles, and uses primers whose 3′ ends encompass the SNP. PCR amplification under stringent conditions is much less efficient in the presence of a mismatch between template and primer, so successful amplification with an SNP-specific primer signals presence of the specific SNP in a sequence (See, Saiki et al. (1986) Nature 324(6093):163-166 and U.S. Pat. No. 5,821,062; 7,052,845 or 7,250,258).

Assembly PCR or Polymerase Cycling Assembly (PCA) is the artificial synthesis of long DNA sequences by performing PCR on a pool of long oligonucleotides with short overlapping segments. The oligonucleotides alternate between sense and antisense directions, and the overlapping segments determine the order of the PCR fragments thereby selectively producing the final long DNA product (See, Stemmer et al. (1995) Gene 164(1):49-53 and U.S. Pat. No. 6,335,160; 7,058,504 or 7,323,336)

Asymmetric PCR is used to preferentially amplify one strand of the original DNA more than the other. It finds use in some types of sequencing and hybridization probing where having only one of the two complementary stands is required. PCR is carried out as usual, but with a great excess of the primers for the chosen strand. Due to the slow amplification later in the reaction after the limiting primer has been used up, extra cycles of PCR are required (See, Innis et al. (1988) Proc Natl Acad Sci U.S.A. 85(24):9436-9440 and U.S. Pat. No. 5,576,180; 6,106,777 or 7,179,600) A recent modification on this process, known as Linear-After-The-Exponential-PCR (LATE-PCR), uses a limiting primer with a higher melting temperature (I′ₘ) than the excess primer to maintain reaction efficiency as the limiting primer concentration decreases mid-reaction (Pierce et al. (2007) Methods Mol. Med. 132:65-85).

Colony PCR uses bacterial colonies, for example E. coli, which can be rapidly screened by PCR for correct DNA vector constructs. Selected bacterial colonies are picked with a sterile toothpick and dabbed into the PCR master mix or sterile water. The PCR is started with an extended time at 95° C. when standard polymerase is used or with a shortened denaturation step at 100° C. and special chimeric DNA polymerase (Pavlov et al. (2006) “Thermostable DNA Polymerases for a Wide Spectrum of Applications: Comparison of a Robust Hybrid TopoTaq to other enzymes”, in Kieleczawa J: DNA Sequencing II: Optimizing Preparation and Cleanup. Jones and Bartlett, pp. 241-257)

Helicase-dependent amplification is similar to traditional PCR, but uses a constant temperature rather than cycling through denaturation and annealing/extension cycles. DNA Helicase, an enzyme that unwinds DNA, is used in place of thermal denaturation (See, Myriam et al. (2004) EMBO reports 5(8):795-800 and U.S. Pat. No. 7,282,328).

Hot-start PCR is a technique that reduces non-specific amplification during the initial set up stages of the PCR. The technique may be performed manually by heating the reaction components to the melting temperature (e.g., 95° C.) before adding the polymerase (Chou et al. (1992) Nucleic Acids Research 20:1717-1723 and U.S. Pat. Nos. 5,576,197 and 6,265,169). Specialized enzyme systems have been developed that inhibit the polymerase's activity at ambient temperature, either by the binding of an antibody (Sharkey et al. (1994) Bio/Technology 12:506-509) or by the presence of covalently bound inhibitors that only dissociate after a high-temperature activation step. Hot-start/cold-finish PCR is achieved with new hybrid polymerases that are inactive at ambient temperature and are instantly activated at elongation temperature.

Intersequence-specific (ISSR) PCR method for DNA fingerprinting that amplifies regions between some simple sequence repeats to produce a unique fingerprint of amplified fragment lengths (Zietkiewicz et al. (1994) Genomics 20(2):176-83).

Inverse PCR is a method used to allow PCR when only one internal sequence is known. This is especially useful in identifying flanking sequences to various genomic inserts. This involves a series of DNA digestions and self ligation, resulting in known sequences at either end of the unknown sequence (Ochman et al. (1988) Genetics 120:621-623 and U.S. Pat. No. 6,013,486; 6,106,843 or 7,132,587).

Ligation-mediated PCR uses small DNA linkers ligated to the DNA of interest and multiple primers annealing to the DNA linkers; it has been used for DNA sequencing, genome walking, and DNA footprinting (Mueller et al. (1988) Science 246:780-786).

Methylation-specific PCR (MSP) is used to detect methylation of CpG islands in genomic DNA (Herman et al. (1996) Proc Natl Acad Sci U.S.A. 93(13):9821-9826 and U.S. Pat. No. 6,811,982; 6,835,541 or 7,125,673). DNA is first treated with sodium bisulfate, which converts unmethylated cytosine bases to uracil, which is recognized by PCR primers as thymine. Two PCRs are then carried out on the modified DNA, using primer sets identical except at any CpG islands within the primer sequences. At these points, one primer set recognizes DNA with cytosines to amplify methylated DNA, and one set recognizes DNA with uracil or thymine to amplify unmethylated DNA. MSP using qPCR can also be performed to obtain quantitative rather than qualitative information about methylation.

Multiplex Ligation-dependent Probe Amplification (MLPA) permits multiple targets to be amplified with only a single primer pair, thus avoiding the resolution limitations of multiplex PCR (see below).

Multiplex-PCR uses of multiple, unique primer sets within a single PCR mixture to produce amplicons of varying sizes specific to different DNA sequences (See, U.S. Pat. No. 5,882,856; 6,531,282 or 7,118,867). By targeting multiple genes at once, additional information may be gained from a single test run that otherwise would require several times the reagents and more time to perform. Annealing temperatures for each of the primer sets must be optimized to work correctly within a single reaction, and amplicon sizes, i.e., their base pair length, should be different enough to form distinct bands when visualized by gel electrophoresis.

Nested PCR increases the specificity of DNA amplification, by reducing background due to non-specific amplification of DNA. Two sets of primers are being used in two successive PCRs. In the first reaction, one pair of primers is used to generate DNA products, which besides the intended target, may still consist of non-specifically amplified DNA fragments. The product(s) are then used in a second PCR with a set of primers whose binding sites are completely or partially different from and located 3′ of each of the primers used in the first reaction (See, U.S. Pat. No. 5,994,006; 7,262,030 or 7,329,493). Nested PCR is often more successful in specifically amplifying long DNA fragments than conventional PCR, but it requires more detailed knowledge of the target sequences.

Overlap-extension PCR is a genetic engineering technique allowing the construction of a DNA sequence with an alteration inserted beyond the limit of the longest practical primer length.

Quantitative PCR (Q-PCR), also known as RQ-PCR, QRT-PCR and RTQ-PCR, is used to measure the quantity of a PCR product following the reaction or in real-time. See, U.S. Pat. No. 6,258,540; 7,101,663 or 7,188,030. Q-PCR is the method of choice to quantitatively measure starting amounts of DNA, cDNA or RNA. Q-PCR is commonly used to determine whether a DNA sequence is present in a sample and the number of its copies in the sample. The method with currently the highest level of accuracy is digital PCR as described in U.S. Pat. No. 6,440,705; U.S. Publication No. 2007/0202525; Dressman et al. (2003) Proc. Natl. Acad. Sci. USA 100(15):8817-8822 and Vogelstein et al. (1999) Proc. Natl. Acad. Sci. USA. 96(16):9236-9241. More commonly, RT-PCR refers to reverse transcription PCR (see below), which is often used in conjunction with Q-PCR. QRT-PCR methods use fluorescent dyes, such as Sybr Green, or fluorophore-containing DNA probes, such as TaqMan, to measure the amount of amplified product in real time.

Reverse Transcription PCR (RT-PCR) is a method used to amplify, isolate or identify a known sequence from a cellular or tissue RNA (See, U.S. Pat. No. 6,759,195; 7,179,600 or 7,317,111). The PCR is preceded by a reaction using reverse transcriptase to convert RNA to cDNA. RT-PCR is widely used in expression profiling, to determine the expression of a gene or to identify the sequence of an RNA transcript, including transcription start and termination sites and, if the genomic DNA sequence of a gene is known, to map the location of exons and introns in the gene. The 5′ end of a gene (corresponding to the transcription start site) is typically identified by an RT-PCR method, named Rapid Amplification of cDNA Ends (RACE-PCR).

Thermal asymmetric interlaced PCR (TAIL-PCR) is used to isolate unknown sequence flanking a known sequence. Within the known sequence TAIL-PCR uses a nested pair of primers with differing annealing temperatures; a degenerate primer is used to amplify in the other direction from the unknown sequence (Liu et al. (1995) Genomics 25(3):674-81).

Touchdown PCR a variant of PCR that aims to reduce nonspecific background by gradually lowering the annealing temperature as PCR cycling progresses. The annealing temperature at the initial cycles is usually a few degrees (3-5° C.) above the Tₙ, of the primers used, while at the later cycles, it is a few degrees (3-5° C.) below the primer Tₘ. The higher temperatures give greater specificity for primer binding, and the lower temperatures permit more efficient amplification from the specific products formed during the initial cycles (Don et al. (1991) Nucl Acids Res 19:4008 and U.S. Pat. No. 6,232,063).

In one embodiment of the invention, probes are labeled with two fluorescent dye molecules to form so-called “molecular beacons” (Tyagi, S, and Kramer, F. R. (1996) Nat. Biotechnol. 14:303-8). Such molecular beacons signal binding to a complementary nucleic acid sequence through relief of intramolecular fluorescence quenching between dyes bound to opposing ends on an oligonucleotide probe. The use of molecular beacons for genotyping has been described (Kostrikis, L. G. (1998) Science 279:1228-9) as has the use of multiple beacons simultaneously (Marras, S. A. (1999) Genet. Anal. 14:151-6). A quenching molecule is useful with a particular fluorophore if it has sufficient spectral overlap to substantially inhibit fluorescence of the fluorophore when the two are held proximal to one another, such as in a molecular beacon, or when attached to the ends of an oligonucleotide probe from about 1 to about 25 nucleotides.

Labeled probes also can be used in conjunction with amplification of a gene of interest. (Holland et al. (1991) Proc. Natl. Acad. Sci. 88:7276-7280). U.S. Pat. No. 5,210,015 by Gelfand et al. describe fluorescence-based approaches to provide real time measurements of amplification products during PCR. Such approaches have either employed intercalating dyes (such as ethidium bromide) to indicate the amount of double-stranded DNA present, or they have employed probes containing fluorescence-quencher pairs (also referred to as the “Taq-Man” approach) where the probe is cleaved during amplification to release a fluorescent molecule whose concentration is proportional to the amount of double-stranded DNA present. During amplification, the probe is digested by the nuclease activity of a polymerase when hybridized to the target sequence to cause the fluorescent molecule to be separated from the quencher molecule, thereby causing fluorescence from the reporter molecule to appear. The Taq-Man approach uses a probe containing a reporter molecule—quencher molecule pair that specifically anneals to a region of a target polynucleotide containing the polymorphism.

Probes can be affixed to surfaces for use as “gene chips.” Such gene chips can be used to detect genetic variations by a number of techniques known to one of skill in the art. In one technique, oligonucleotides are arrayed on a gene chip for determining the DNA sequence of a by the sequencing by hybridization approach, such as that outlined in U.S. Pat. Nos. 6,025,136 and 6,018,041. The probes of the invention also can be used for fluorescent detection of a genetic sequence. Such techniques have been described, for example, in U.S. Pat. Nos. 5,968,740 and 5,858,659. A probe also can be affixed to an electrode surface for the electrochemical detection of nucleic acid sequences such as described by Kayem et al. U.S. Pat. No. 5,952,172 and by Kelley, S. O. et al. (1999) Nucleic Acids Res. 27:4830-4837.

This invention also provides for a prognostic panel of genetic markers selected from, but not limited to the genetic polymorphisms identified herein. The prognostic panel comprises probes or primers or a microarray that can be used to amplify and/or for determining the molecular structure of the polymorphisms identified herein. The probes or primers can be attached or supported by a solid phase support such as, but not limited to a gene chip or microarray. The probes or primers can be detectably labeled. This aspect of the invention is a means to identify the genotype of a patient sample for the genes of interest identified above.

In one aspect, the panel contains the herein identified probes or primers as wells as other probes or primers. In a alternative aspect, the panel includes one or more of the above noted probes or primers and others. In a further aspect, the panel consist only of the above-noted probes or primers.

Primers or probes can be affixed to surfaces for use as “gene chips” or “microarray.” Such gene chips or microarrays can be used to detect genetic variations by a number of techniques known to one of skill in the art. In one technique, oligonucleotides are arrayed on a gene chip for determining the DNA sequence of a by the sequencing by hybridization approach, such as that outlined in U.S. Pat. Nos. 6,025,136 and 6,018,041. The probes of the invention also can be used for fluorescent detection of a genetic sequence. Such techniques have been described, for example, in U.S. Pat. Nos. 5,968,740 and 5,858,659. A probe also can be affixed to an electrode surface for the electrochemical detection of nucleic acid sequences such as described by Kayem et al. U.S. Pat. No. 5,952,172 and by Kelley et al. (1999) Nucleic Acids Res. 27:4830-4837.

Various “gene chips” or “microarray” and similar technologies are know in the art. Examples of such include, but are not limited to LabCard (ACLARA Bio Sciences Inc.); GeneChip (Affymetric, Inc); LabChip (Caliper Technologies Corp); a low-density array with electrochemical sensing (Clinical Micro Sensors); LabCD System (Gamera Bioscience Corp.); Omni Grid (Gene Machines); Q Array (Genetix Ltd.); a high-throughput, automated mass spectrometry systems with liquid-phase expression technology (Gene Trace Systems, Inc.); a thermal jet spotting system (Hewlett Packard Company); Hyseq HyChip (Hyseq, Inc.); BeadArray (Illumina, Inc.); GEM (Incyte Microarray Systems); a high-throughput microarraying system that can dispense from 12 to 64 spots onto multiple glass slides (Intelligent Bio-Instruments); Molecular Biology Workstation and NanoChip (Nanogen, Inc.); a microfluidic glass chip (Orchid biosciences, Inc.); BioChip Arrayer with four PiezoTip piezoelectric drop-on-demand tips (Packard Instruments, Inc.); FlexJet (Rosetta Inpharmatic, Inc.); MALDI-TOF mass spectrometer (Sequnome); ChipMaker 2 and ChipMaker 3 (TeleChem International, Inc.); and GenoSensor (Vysis, Inc.) as identified and described in Heller (2002) Annu Rev. Biomed. Eng. 4:129-153. Examples of “Gene chips” or a “microarray” are also described in U.S. Patent Publ. Nos.: 2007/0111322, 2007/0099198, 2007/0084997, 2007/0059769 and 2007/0059765 and U.S. Pat. Nos. 7,138,506, 7,070,740, and 6,989,267.

In one aspect, “gene chips” or “microarrays” containing probes or primers for the gene of interest are provided alone or in combination with other probes and/or primers. A suitable sample is obtained from the patient extraction of genomic DNA, RNA, or any combination thereof and amplified if necessary. The DNA or RNA sample is contacted to the gene chip or microarray panel under conditions suitable for hybridization of the gene(s) of interest to the probe(s) or primer(s) contained on the gene chip or microarray. The probes or primers may be detectably labeled thereby identifying the polymorphism in the gene(s) of interest. Alternatively, a chemical or biological reaction may be used to identify the probes or primers which hybridized with the DNA or RNA of the gene(s) of interest. The genetic profile of the patient is then determined with the aid of the aforementioned apparatus and methods.

### Nucleic Acids

In one aspect, the nucleic acid sequences of the gene of interest, or portions thereof, can be the basis for probes or primers, e.g., in methods for determining expression level of the gene of interest or the allelic variant of a polymorphic region of a gene of interest identified in the experimental section below. Thus, they can be used in the methods of the invention to determine which therapy is most likely to treat an individual's cancer.

The methods of the invention can use nucleic acids isolated from vertebrates. In one aspect, the vertebrate nucleic acids are mammalian nucleic acids. In a further aspect, the nucleic acids used in the methods of the invention are human nucleic acids.

Primers for use in the methods of the invention are nucleic acids which hybridize to a nucleic acid sequence which is adjacent to the region of interest or which covers the region of interest and is extended. A primer can be used alone in a detection method, or a primer can be used together with at least one other primer or probe in a detection method. Primers can also be used to amplify at least a portion of a nucleic acid. Probes for use in the methods of the invention are nucleic acids which hybridize to the gene of interest and which are not further extended. For example, a probe is a nucleic acid which hybridizes to the gene of interest, and which by hybridization or absence of hybridization to the DNA of a subject will be indicative of the identity of the allelic variant of the expression levels of the gene of interest. Primers and/or probes for use in the methods can be provided as isolated single stranded oligonucleotides or alternatively, as isolated double stranded oligonucleotides.

In one embodiment, primers comprise a nucleotide sequence which comprises a region having a nucleotide sequence which hybridizes under stringent conditions to about: 6, or alternatively 8, or alternatively 10, or alternatively 12, or alternatively 25, or alternatively 30, or alternatively 40, or alternatively 50, or alternatively 75 consecutive nucleotides of the gene of interest.

Primers can be complementary to nucleotide sequences located close to each other or further apart, depending on the use of the amplified DNA. For example, primers can be chosen such that they amplify DNA fragments of at least about 10 nucleotides or as much as several kilobases. Preferably, the primers of the invention will hybridize selectively to nucleotide sequences located about 100 to about 1000 nucleotides apart.

For amplifying at least a portion of a nucleic acid, a forward primer (i.e., 5′ primer) and a reverse primer (i.e., 3′ primer) will preferably be used. Forward and reverse primers hybridize to complementary strands of a double stranded nucleic acid, such that upon extension from each primer, a double stranded nucleic acid is amplified.

Yet other preferred primers of the invention are nucleic acids which are capable of selectively hybridizing to the gene. Thus, such primers can be specific for the gene of interest sequence, so long as they have a nucleotide sequence which is capable of hybridizing to the gene of interest.

The probe or primer may further comprises a label attached thereto, which, e.g., is capable of being detected, e.g. the label group is selected from amongst radioisotopes, fluorescent compounds, enzymes, and enzyme co-factors.

Additionally, the isolated nucleic acids used as probes or primers may be modified to become more stable. Exemplary nucleic acid molecules which are modified include phosphoramidate, phosphothioate and methylphosphonate analogs of DNA (see also U.S. Pat. Nos. 5,176,996; 5,264,564 and 5,256,775).

The nucleic acids used in the methods of the invention can also be modified at the base moiety, sugar moiety, or phosphate backbone, for example, to improve stability of the molecule. The nucleic acids, e.g., probes or primers, may include other appended groups such as peptides (e.g., for targeting host cell receptors in vivo), or agents facilitating transport across the cell membrane. See, e.g., Letsinger et al. (1989) Proc. Natl. Acad. Sci. U.S.A. 86:6553-6556; Lemaitre et al. (1987) Proc. Natl. Acad. Sci. 84:648-652; and PCT Publ. No. WO 88/09810, published Dec. 15, 1988), hybridization-triggered cleavage agents, (see, e.g., Krol et al. (1988) BioTechniques 6:958-976) or intercalating agents (see, e.g., Zon (1988) Pharm. Res. 5:539-549. To this end, the nucleic acid used in the methods of the invention may be conjugated to another molecule, e.g., a peptide, hybridization triggered cross-linking agent, transport agent, hybridization-triggered cleavage agent, etc.

The isolated nucleic acids used in the methods of the invention can also comprise at least one modified sugar moiety selected from the group including but not limited to arabinose, 2-fluoroarabinose, xylulose, and hexose or, alternatively, comprise at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

The nucleic acids, or fragments thereof, to be used in the methods of the invention can be prepared according to methods known in the art and described, e.g., in Sambrook et al. (2001) supra. For example, discrete fragments of the DNA can be prepared and cloned using restriction enzymes. Alternatively, discrete fragments can be prepared using the Polymerase Chain Reaction (PCR) using primers having an appropriate sequence under the manufacturer's conditions, (described above).

Oligonucleotides can be synthesized by standard methods known in the art, e.g. by use of an automated DNA synthesizer (such as are commercially available from Biosearch, Applied Biosystems, etc.). As examples, phosphorothioate oligonucleotides can be synthesized by the method of Stein et al. (1988) Nucl. Acids Res. 16:3209, methylphosphonate oligonucleotides can be prepared by use of controlled pore glass polymer supports. Sarin et al. (1988) Proc. Natl. Acad. Sci. U.S.A. 85:7448-7451.

### Kits

Also provided is a kit for use in determining if a cancer patient treated with surgery is more likely to experience tumor recurrence, comprising, or alternatively consisting essentially of, or yet further consisting of, suitable primers or probes or a microarray for determining at least one polymorphism of the group PAR-1-506 ins/del or EGF+61 A>G, and instructions for use therein.

In one aspect, the patient suffered at least one cancer of the type of the group: esophageal adenocarcinoma, lung cancer, breast cancer, head and neck cancer, ovarian cancer, metastatic or non-metastatic rectal cancer, metastatic or non-metastatic colon cancer, metastatic or non-metastatic colorectal cancer, or non-small cell lung cancer (NSCLC) before being treated with surgery. In a particular aspect, the patient suffered at least esophageal adenocarcinoma before being treated with surgery.

In one aspect of the above noted methods, the cancer patient, after surgical resection, underwent chemotherapy or radiotherapy. Non-limiting examples of chemotherapy include administration of anti-VEGF antibodies such as Bevacizumab, anti-EGFR antibodies such as Cetuximab, topoisomerase inhibitors such as Irinotecan (CTP-11), platinum drugs such as carboplatin or oxaliplatin, mitotic inhibitors such as paclitaxel, pyrimidine antimetabolites such as 5-FU or capecitabin, or FOLFOX or XELOX.

Suitable patient samples in the methods include, but are not limited to a sample comprises, or alternatively consisting essentially of, or yet further consisting of, at least one of a tumor cell, a normal cell adjacent to a tumor, a normal cell corresponding to the tumor tissue type, a blood cell, a peripheral blood lymphocyte, or combinations thereof. The samples can be at least one of a fixed tissue, a frozen tissue, a biopsy tissue, a resection tissue, a microdissected tissue, or combinations thereof.

Any suitable method for identifying the genotype in the patient sample can be used and the inventions described herein are not to be limited to these methods. For the purpose of illustration only, the genotype is determined by a method comprising, or alternatively consisting essentially of, or yet further consisting of, hybridization, PCR or more specifically, PCR-RFLP or microarray. These methods as well as equivalents or alternatives thereto are described herein.

The methods are useful in the assistance of an animal, a mammal or yet further a human patient. For the purpose of illustration only, a mammal includes but is not limited to a simian, a murine, a bovine, an equine, a porcine or an ovine.

As set forth herein, the invention provides diagnostic methods for determining the polymorphic region of the gene of interest. In some embodiments, the methods use probes or primers comprising nucleotide sequences which are complementary to the gene of interest.

The kit can comprise at least one probe or primer which is capable of specifically hybridizing to the gene of interest and instructions for use. The kits preferably comprise at least one of the above described nucleic acids. Preferred kits for amplifying at least a portion of the gene of interest comprise two primers, at least one of which is capable of hybridizing to the allelic variant sequence. Such kits are suitable for detection of genotype by, for example, fluorescence detection, by electrochemical detection, or by other detection.

Oligonucleotides, whether used as probes or primers, contained in a kit can be detectably labeled. Labels can be detected either directly, for example for fluorescent labels, or indirectly. Indirect detection can include any detection method known to one of skill in the art, including biotin-avidin interactions, antibody binding and the like. Fluorescently labeled oligonucleotides also can contain a quenching molecule. Oligonucleotides can be bound to a surface. In one embodiment, the preferred surface is silica or glass. In another embodiment, the surface is a metal electrode.

Yet other kits of the invention comprise at least one reagent necessary to perform the assay. For example, the kit can comprise an enzyme. Alternatively the kit can comprise a buffer or any other necessary reagent.

Conditions for incubating a nucleic acid probe with a test sample depend on the format employed in the assay, the detection methods used, and the type and nature of the nucleic acid probe used in the assay. One skilled in the art will recognize that any one of the commonly available hybridization, amplification or immunological assay formats can readily be adapted to employ the nucleic acid probes for use in the present invention. Examples of such assays can be found in Chard, T. (1986) AN INTRODUCTION TO RADIOIMMUNOASSAY AND RELATED TECHNIQUES Elsevier Science Publishers, Amsterdam, The Netherlands; Bullock, G. R. et al., TECHNIQUES IN IMMUNOCYTOCHEMISTRY Academic Press, Orlando, Fla. Vol. 1 (1982), Vol. 2 (1983), Vol. 3 (1985); Tijssen, P. (1985) PRACTICE AND THEORY OF IMMUNOASSAYS: LABORATORY TECHNIQUES IN BIOCHEMISTRY AND MOLECULAR BIOLOGY, Elsevier Science Publishers, Amsterdam, The Netherlands.

The test samples used in the diagnostic kits include cells, protein or membrane extracts of cells, or biological fluids such as sputum, blood, serum, plasma, or urine. The test samples may also be a tumor cell, a normal cell adjacent to a tumor, a normal cell corresponding to the tumor tissue type, a blood cell, a peripheral blood lymphocyte, or combinations thereof. The test sample used in the above-described method will vary based on the assay format, nature of the detection method and the tissues, cells or extracts used as the sample to be assayed. Methods for preparing protein extracts or membrane extracts of cells are known in the art and can be readily adapted in order to obtain a sample which is compatible with the system utilized.

The kits can include all or some of the positive controls, negative controls, reagents, primers, sequencing markers, probes and antibodies described herein for determining the subject's genotype in the polymorphic region of the gene of interest.

As amenable, these suggested kit components may be packaged in a manner customary for use by those of skill in the art. For example, these suggested kit components may be provided in solution or as a liquid dispersion or the like.

### Other Uses for the Nucleic Acids of the Invention

The identification of the polymorphic region or the expression level of the gene of interest can also be useful for identifying an individual among other individuals from the same species. For example, DNA sequences can be used as a fingerprint for detection of different individuals within the same species. Thompson, J. S, and Thompson, eds., (1991) GENETICS IN MEDICINE, W B Saunders Co., Philadelphia, Pa. This is useful, e.g., in forensic studies.

The invention now being generally described, it will be more readily understood by reference to the following example which is included merely for purposes of illustration of certain aspects and embodiments of the present invention, and are not intended to limit the invention.

### EXPERIMENTAL DETAILS

### Example 1

### Patients and Methods

### Patients

Two hundred and thirty nine (n=239) patients with localized (stage Ib-III) esophageal adenocarcinoma who were treated with surgery alone at the University of Southern California/University Hospital (USC/UH) or the Los Angeles County/University of Southern California Medical Center (LAC/USCMC), between 1992 and 2007, were eligible for the present study. This study was conducted at the USC/UH and approved by the Institutional Review Board of the University of Southern California for Medical Sciences. Patient data were collected through chart review. All patients involved in the study signed informed consent for the analysis of molecular correlates. Detailed clinic-pathologic characteristics are shown in Table 1.

### Genotyping

Formalin-fixed paraffin-embedded tumor samples were collected and genomic DNA was extracted using the QIAamp extraction kit (Qiagen, CA, USA) according to the manufacturer's protocol. The majority of the samples were tested using polymerase chain reaction restriction fragment length polymorphism (PCR-RFLP) technique. Briefly, forward and reverse primers were used for PCR amplification, PCR products were digested by restriction enzymes (New England Biolab, Massachussetts, USA) and alleles were separated using a 4% NuSieve ethidium bromide stained agarose gel. Forward and reverse primer, restriction enzymes and annealing temperatures are listed in Table 2.

The dinucleotide polymorphisms (Table 3) were determined with 5′-end ³³P γATP labeled PCR protocol with a few modifications. In summary: DNA template, dNTPs, 5′-end ³³P γATP labeled primer, unlabelled complementary primer, Taq Polymerase (Perkin Elmer Inc, Connecticut, USA) and PCR Buffer were used together in a final PCR. The reaction was carried out and the reaction products were separated using a 6% denaturing polyacrylamid DNA sequencing gel, which then was vacuum-blotted for 1 h at 80° C. and exposed to a XAR film (Eastman-Kodak Co.; New York, USA) overnight. The exact number of repeats was confirmed by direct sequencing.

TABLE 1
Clinico-pathological characteristics and clinical outcome in patients with resected esophageal adenocarcinoma

| | | Time to recurrence | | | | | Overall Survival | | |
|---|---|---|---|---|---|---|---|---|---|
| | | | | Probability ± | | | | Probability ± | |
| | | Median time to | | SE* of | P | Median overall | | SE* of | P |
| | | recurrence, yrs | Relative risk | 5-year | value | survival, yrs | Relative risk | 5-year | value |
| | N | (95% CI) | (95% CI) | recurrence | † | (95% CI) | (95% CI) | survival | † |
| Sex | | | | | 0.99 | | | | 0.35 |
| Male | 203 | 2.8 (1.9, 4.2) | 1 | 0.60 ± 0.04 | | 2.8 (2.1, 3.7) | 1 | 0.34 ± 0.04 | |
| Female | 36 | 2.1 (1.7, 9.8+) | 1.00 (0.61, 1.64) | 0.67 ± 0.10 | | 5.2 (2.0, 8.1) | 0.80 (0.50, 1.29) | 0.52 ± 0.10 | |
| Age, years | | | | | 0.20 | | | | <0.001 |
| <60 | 92 | 3.3 (2.7, 11.0+) | 1 | 0.56 ± 0.07 | | 4.6 (3.1, 9.0) | 1 | 0.48 ± 0.07 | |
| 60-69 | 64 | 3.2 (1.9, 9.8+) | 1.01 (0.64, 1.61) | 0.59 ± 0.07 | | 3.5 (2.4, 5.3) | 1.30 (0.84, 2.00) | 0.40 ± 0.07 | |
| ≧70 | 83 | 1.9 (1.3, 4.2) | 1.42 (0.92, 2.18) | 0.70 ± 0.07 | | 1.5 (0.9, 2.2) | 2.16 (1.46, 3.21) | 0.23 ± 0.06 | |
| Race | | | | | 0.90 | | | | 0.60 |
| Caucasian | 221 | 2.7 (1.9, 4.2) | 1 | 0.62 ± 0.04 | | 2.8 (2.2, 3.7) | 1 | 0.37 ± 0.04 | |
| Asian | 5 | 3.2+ (0.1, 3.2+) | 0.90 (0.22, 3.65) | 0.20 ± 0.18 | | 3.3+ (0.2, 3.3+) | 1.25 (0.40, 3.93) | 0.60 ± 0.24 | |
| Hispanic | 13 | 5.5+ (1.0, 5.5+) | 0.83 (0.36, 1.89) | 0.50 ± 0.15 | | 4.2 (1.2, 5.5+) | 0.68 (0.30, 1.54) | 0.40 ± 0.15 | |
| Stage | | | | | <0.001 | | | | <0.001 |
| Ib | 34 | 10.3+ | 1 | 0.19 ± 0.08 | | 10.3+ (4.1, 10.3+) | 1 | 0.71 ± 0.11 | |
| IIa | 36 | 6.2+ (3.3, 6.2+) | 2.61 (0.90, 7.56) | 0.44 ± 0.12 | | 6.4 (2.2, 14.3+) | 2.07 (0.91, 4.71) | 0.55 ± 0.11 | |
| IIb | 33 | 2.4 (1.6, 4.4) | 5.46 (2.07, 14.4) | 0.71 ± 0.09 | | 5.0 (1.9, 5.9) | 2.71 (1.24, 5.92) | 0.51 ± 0.10 | |
| III | 136 | 1.6 (1.0, 1.9) | 7.33 (3.00, 17.9) | 0.75 ± 0.05 | | 2.0 (1.4, 2.6) | 4.58 (2.32, 9.02) | 0.20 ± 0.04 | |
| T-category | | | | | <0.001 | | | | <0.001 |
| T1 | 43 | 10.3+ | 1 | 0.25 ± 0.08 | | 10.3+ (4.1, 10.3+) | 1 | 0.68 ± 0.09 | |
| T2 | 44 | 4.0 (2.8, 6.2) | 2.53 (1.15, 5.55) | 0.64 ± 0.11 | | 5.0 (3.6, 6.4) | 2.07 (1.06, 4.04) | 0.55 ± 0.09 | |
| T3 ‡ | 151 | 1.7 (1.3, 2.1) | 4.55 (2.30, 9.00) | 0.74 ± 0.05 | | 2.0 (1.5, 2.6) | 3.61 (2.03, 6.40) | 0.22 ± 0.04 | |
| T4 ‡ | 1 | | | | | | | | |
| N-category | | | | | <0.001 | | | | <0.001 |
| Negative | 69 | 10.3+ (6.2, 10.3+) | 1 | 0.30 ± 0.07 | | 11.6 (4.1, 14.3+) | 1 | 0.63 ± 0.08 | |
| 1-2 positive | 48 | 2.3 (1.4, 13.2+) | 2.70 (1.43, 5.10) | 0.57 ± 0.08 | | 3.7 (1.9, 13.2+) | 1.72 (1.00, 2.95) | 0.47 ± 0.08 | |
| 3-7 Positive | 55 | 2.1 (1.0, 4.2) | 3.58 (1.95, 6.56) | 0.74 ± 0.08 | | 3.3 (2.0, 4.5) | 2.42 (1.46, 4.01) | 0.29 ± 0.07 | |
| ≧8 positive | 67 | 1.3 (0.6, 1.8) | 5.89 (3.25, 10.7) | 0.94 ± 0.05 | | 1.4 (0.8, 1.8) | 4.40 (2.69, 7.19) | 0.08 ± 0.06 | |
| Differen- | | | | | 0.022 | | | | <0.001 |
| tiation | | | | | | | | | |
| Well/ | 102 | 3.5 (2.3, 13.2+) | 1 | 0.55 ± 0.06 | | 4.1 (3.1, 5.8) | 1 | 0.42 ± 0.06 | |
| moderate | | | | | | | | | |
| Poor | 135 | 1.9 (1.4, 3.8) | 1.54 (1.06, 2.25) | 0.67 ± 0.05 | | 2.0 (1.4, 2.9) | 1.79 (1.27, 2.53) | 0.34 ± 0.05 | |
| Type of | | | | | 0.10 | | | | 0.003 |
| surgery | | | | | | | | | |
| En Bloc | 137 | 3.8 (2.4, 6.2) | 1 | 0.58 ± 0.05 | | 4.1 (3.1, 5.3) | 1 | 0.45 ± 0.05 | |
| Transhiatal | 102 | 1.9 (1.4, 3.3) | 1.36 (0.94, 1.97) | 0.66 ± 0.06 | | 2.0 (1.4, 2.8) | 1.63 (1.18, 2.27) | 0.26 ± 0.05 | |

*Greenwood SE
+Estimates were not reached
† Based on log-rank test
‡ Grouped together for the estimates of relative risk and probability ± SE of 3-year recurrence

TABLE 2
Primer Sequences, Annealing Temperatures, and Restriction Enzyme

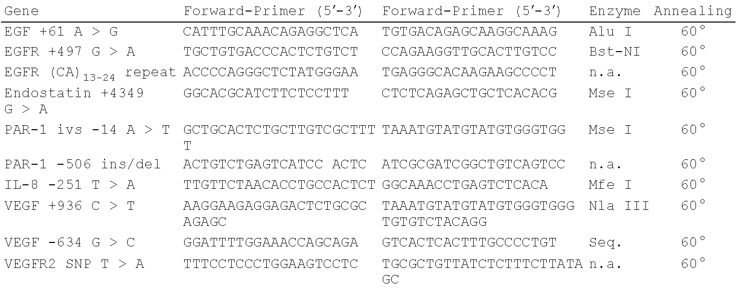

Abbreviations:
EGF = epidermal growth factor;
EGFR = epidermal growth factor receptor;
PAR-1 = protease activated receptor;
IL-8 = Interleukin-8;
VEGF = vascular endothelial growth factor;
VEGFR2 = vascular endothelial growth factor receptor 2

TABLE 3
Polymorphisms and clinical outcome in patients with resected esophageal adenocarcinoma

| | Time to recurrence | | | | | Overall Survival | | | |
|---|---|---|---|---|---|---|---|---|---|
| | | | | Probability ± | | | | Probability ± | |
| | | Median time to | | SE* of | P | Median overall | | SE* of | P |
| | | recurrence, yrs | Relative risk | 5-year | value | survival, yrs | Relative risk | 5-year | value |
| | N | (95% CI) | (95% CI) | recurrence | † | (95% CI) | (95% CI) | recurrence | † |
| EGF +61 A > G | | | | | 0.034 | | | | 0.19 |
| (rs4444903) | | | | | | | | | |
| A/A | 66 | 1.8 (0.8, 2.8) | 1 | 0.76 ± 0.07 | | 2.0 (1.2, 3.7) | 1 | 0.27 ± 0.07 | |
| A/G | 128 | 3.8 (2.2, 13.2+) | 0.58 (0.38, 0.88) | 0.54 ± 0.06 | | 3.5 (2.6, 5.0) | 0.71 (0.49, 1.03) | 0.41 ± 0.05 | |
| G/G | 44 | 3.2 (1.3, 6.2+) | 0.75 (0.45, 1.27) | 0.63 ± 0.09 | | 3.3 (1.3, 6.7) | 0.76 (0.47, 1.24) | 0.36 ± 0.08 | |
| EGFR +497 G > A | | | | | 0.97 | | | | 0.85 |
| (rs11543848) | | | | | | | | | |
| G/G | 124 | 3.3 (1.8, 6.2) | 1 | 0.60 ± 0.06 | | 3.6 (2.0, 5.2) | 1 | 0.40 ± 0.05 | |
| G/A | 90 | 2.8 (1.9, 4.9) | 1.02 (0.69, 1.50) | 0.63 ± 0.06 | | 2.9 (2.1, 4.4) | 1.07 (0.75, 1.51) | 0.36 ± 0.06 | |
| A/A | 20 | 1.7 (1.3, 5.2+) | 1.09 (0.55, 2.13) | 0.58 ± 0.12 | | 2.9 (0.7, 5.2+) | 1.17 (0.63, 2.17) | 0.26 ± 0.14 | |
| EGFR (CA)₁₃₋₂₄ | | | | | 0.79 | | | | 0.55 |
| repeat | | | | | | | | | |
| (rs45608036) | | | | | | | | | |
| Both (CA)ₙ < 20 | 108 | 2.8 (1.8, 9.8+) | 1 | 0.62 ± 0.06 | | 3.6 (2.2, 4.6) | 1 | 0.38 ± 0.05 | |
| One (CA)ₙ ≧ 20 | 96 | 4.2 (1.8, 6.2) | 1.01 (0.67, 1.50) | 0.58 ± 0.07 | | 2.9 (2.0, 5.9) | 0.96 (0.67, 1.38) | 0.42 ± 0.06 | |
| Both (CA)ₙ ≧ 20 | 31 | 2.1 (1.5, 9.8+) | 1.21 (0.69, 2.12) | 0.69 ± 0.11 | | 2.6 (1.1, 3.6) | 1.27 (0.77, 2.10) | 0.24 ± 0.10 | |
| Endostatin +4349 | | | | | 0.13 | | | | 0.51 |
| G > A | | | | | | | | | |
| (rs12483377) | | | | | | | | | |
| G/G | 214 | 2.7 (1.8, 4.0) | 1 | 0.63 ± 0.04 | | 2.9 (2.2, 3.7) | 1 | 0.36 ± 0.04 | |
| G/A | 24 | 3.8 (2.2, 9.8+) | 0.58 (0.28, 1.18) | 0.50 ± 0.14 | | 4.1 (0.9, 9.8+) | 0.83 (0.47, 1.46) | 0.45 ± 0.11 | |
| PAR-1 ivs −14 | | | | | 0.74 | | | | 0.91 |
| A > T | | | | | | | | | |
| (rs168753) | | | | | | | | | |
| A/A | 160 | 2.2 (1.8, 4.2) | 1 | 0.64 ± 0.05 | | 2.7 (2.0, 3.7) | 1 | 0.36 ± 0.04 | |
| A/T | 62 | 3.2 (1.8, 9.7+) | 0.87 (0.56, 1.33) | 0.57 ± 0.08 | | 3.9 (2.1, 4.6) | 0.92 (0.63, 1.34) | 0.34 ± 0.07 | |
| T/T | 12 | 7.1 (0.8, 7.1+) | 0.79 (0.32, 1.96) | 0.47 ± 0.16 | | 1.8 (0.5, 7.1+) | 0.99 (0.46, 2.13) | 0.38 ± 0.14 | |
| PAR-1 −506 ins/del | | | | | 0.003 | | | | 0.083 |
| (rs11267092) | | | | | | | | | |
| Del/del | 127 | 4.0 (2.7, 13.2+) | 1 | 0.54 ± 0.06 | | 3.6 (2.6, 4.6) | 1 | 0.40 ± 0.05 | |
| Ins/del | 89 | 1.9 (1.1, 3.1) | 1.70 (1.15, 2.50) | 0.70 ± 0.06 | | 2.3 (1.5, 3.9) | 1.36 (0.96, 1.93) | 0.31 ± 0.06 | |
| Ins/ins | 21 | 0.9 (0.5, 3.8) | 2.41 (1.25, 4.65) | 0.81 ± 0.15 | | 2.4 (0.7, 5.3) | 1.68 (0.93, 3.05) | 0.34 ± 0.12 | |
| I1-8 −251 T > A | | | | | 0.58 | | | | 0.82 |
| (rs4073) | | | | | | | | | |
| T/T | 66 | 2.8 (2.0, 4.2) | 1 | 0.68 ± 0.08 | | 3.3 (2.1, 4.1) | 1 | 0.25 ± 0.07 | |
| T/A | 117 | 3.1 (1.8, 11.0+) | 0.90 (0.58, 1.39) | 0.57 ± 0.06 | | 2.8 (2.0, 4.6) | 0.90 (0.62, 1.32) | 0.39 ± 0.05 | |
| A/A | 53 | 1.9 (1.0, 6.2) | 1.15 (0.69, 1.91) | 0.67 ± 0.09 | | 4.6 (1.5, 8.1) | 0.88 (0.55, 1.40) | 0.49 ± 0.08 | |
| VEGF +936 C > T | | | | | 0.82 | | | | 0.81 |
| (rs3025039) | | | | | | | | | |
| C/C | 173 | 2.7 (1.8, 4.9) | 1 | 0.60 ± 0.05 | | 2.8 (2.1, 3.9) | 1 | 0.36 ± 0.04 | |
| C/T ‡ | 63 | 3.2 (1.8, 4.4) | 1.05 (0.69, 1.59) | 0.69 ± 0.08 | | 3.6 (1.4, 5.2) | 1.05 (0.72, 1.52) | 0.38 ± 0.07 | |
| T/T ‡ | 2 | | | | | | | | |
| VEGF −634 G > C | | | | | 0.62 | | | | 0.13 |
| (rs2010963) | | | | | | | | | |
| G/G | 110 | 2.8 (1.8, 11.0+) | 1 | 0.62 ± 0.06 | | 3.6 (2.2, 4.5) | 1 | 0.39 ± 0.06 | |
| G/C | 98 | 1.9 (1.4, 6.2) | 1.13 (0.76, 1.67) | 0.62 ± 0.06 | | 2.2 (1.5, 2.9) | 1.30 (0.92, 1.85) | 0.31 ± 0.05 | |
| C/C | 30 | 4.2 (2.0, 10.3+) | 0.85 (0.47, 1.54) | 0.58 ± 0.11 | | 3.6 (2.1, 14.3+) | 0.81 (0.47, 1.40) | 0.50 ± 0.10 | |
| VEGFR2 T > A | | | | | 0.52 | | | | 0.54 |
| (rs1870377) | | | | | | | | | |
| A/A | 137 | 3.1 (1.9, 4.4) | 1 | 0.61 ± 0.06 | | 3.1 (2.2, 4.5) | 1 | 0.39 ± 0.05 | |
| A/T | 89 | 2.4 (1.3, 6.2) | 1.09 (0.74, 1.61) | 0.62 ± 0.06 | | 2.9 (1.7, 4.1) | 0.96 (0.68, 1.36) | 0.34 ± 0.06 | |
| T/T | 12 | 1.8 (0.5, 5.8+) | 1.56 (0.71, 3.41) | 0.68 ± 0.15 | | 2.0 (0.4, 5.8+) | 1.45 (0.70, 3.01) | 0.25 ± 0.15 | |

*Greenwood SE
+Estimates were not reached;
† Based on log-rank test
‡ Grouped together for the estimates of relative risk and probability ± SE of 3-year recurrence

### Statistical Analysis

The primary endpoint in this study was time to tumor recurrence (TTR) in localized esophageal adenocarcinoma patients, which was defined as the period from the date of surgery to the date of first recurrence or until last contact if the patient was free of any tumor recurrence at the time of last contact. If a patient had not recurred, then TTR was censored at the time of death or at the last follow-up. The secondary endpoint was overall survival that was defined as the period from surgery to death from any cause or the last contact if the patient was alive. The associations of time to tumor recurrence and overall survival with patient's clinico-pathological characteristics (age, sex, race, tumor grade, T-stage, N-stage and type of surgery) were assessed using univariate survival analyses (log-rank test).

Distribution of the alleles of all polymorphisms was tested for deviation from Hardy-Weinberg equilibrium (HWE). The allele frequencies observed were within the probability limits of HWE in Caucasian patients (P<0.05, exact test for HWE). The association between each polymorphism and time to recurrence and overall survival was examined using Kaplan-Meier curves and log-rank test. The distributions of polymorphisms across demographic characteristics were examined using Fisher's exact test. In the univariate survival analysis, the Pike estimate of relative risk (RR) and its associated 95% confidence interval (95% CI) was based on the log-rank test.

The Cox proportional hazards regression model with stratification factors (race, T-category, N-Category) was fitted to re-evaluate the association between polymorphisms and time to recurrence considering the imbalances in the distributions of baseline characteristics. P values of the log-likelihood ratio test were obtained from the modeling. All statistical tests were two-sided. Analyses were performed using the SAS statistical package version 9.1 (SAS Institute Inc. Cary, N.C., USA).

### Results

A total of 239 patients with localized adenocarcinoma of the esophagus (stage Ib-III) were included in this analysis: 203 men (85%) and 36 women (15%) with a median age of 64 years (range: 25-91 years). There were 221 Caucasian (93%), 13 Hispanic (5%), and 5 Asian (2%) study participants. All patients were diagnosed with localized esophageal adenocarcinoma during the years of 1992 and 2007. The median follow-up was 3.2 years. 114 out of 239 patients had tumor recurrence, with a probability of 3-year recurrence of 0.51±0.04. The median time to recurrence was 2.8 years (95% CI::1.9-4.2). 101 out of 114 (89%) patients showed recurrent disease within the first 3 years after surgery. Excluding 4 patients without information on the site of recurrence, 63 patients showed systemic recurrence (57%), 27 patients (25%) displayed recurrence in distant notes, 11 patients (10%) had locoregional recurrence, and 9 patients (8%) had both locoregional and systemic recurrence. 144 out of 239 patients have died and the median overall survival (OS) for the cohort is 2.9 years (95% CI: 2.2-3.9). Tumor stage (p<0.001), T-category (p<0.001), N-category (p<0.001), and tumor differentiation (TTR; p=0.022) (OS; p<0.001), were significantly associated with TTR and OS, whereas the age of the patient (p<0.001) and type surgery performed (p=0.003) were additionally associated with OS but not with TTR. Significant associations between other demographic and clinico-pathological variables and TTR and OS were not observed. Detailed clinico-pathological characteristics are shown in Table 1.

Polymorphisms of PAR-1 and EGF were not associated with demographic (age, gender and ethnicity), clinical (type of surgery), or pathologic characteristics (tumor grade, T- and N-category) (data not shown).

### PAR-1-506 ins/del (rs11267092) and TTR in Localized Esophageal Adenocarcinoma

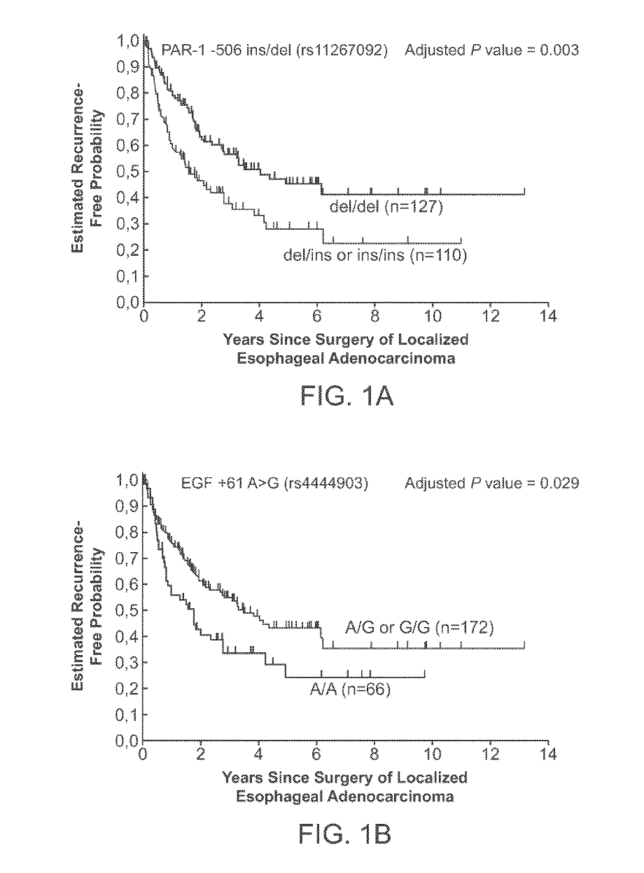

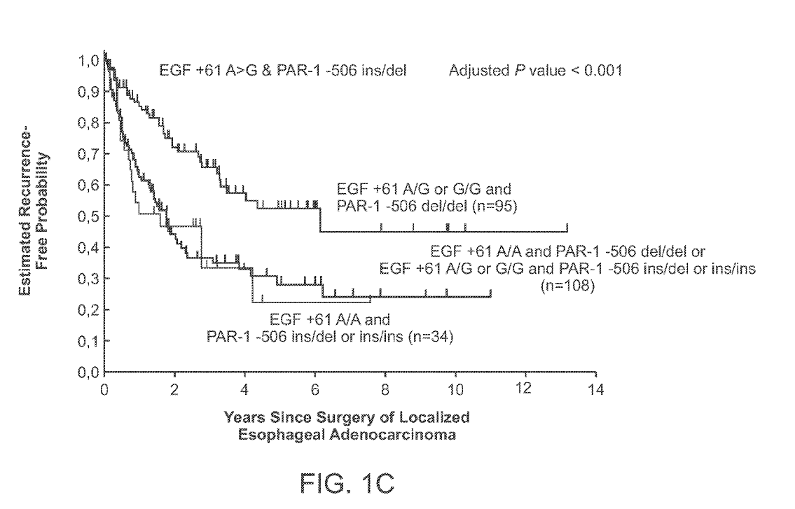

Genotyping for PAR-1-506 ins/del was successful in 237 (99%) of 239 cases. In the other 2 patients (1%) genotyping was not successful, because of limited quantity and quality of extracted genomic DNA. Fifty-four percent (127/237) of patients were homozygous for PAR-1-506 deletion allele (del/del), 38% (89/237) were heterozygous (ins/del), and 8% (21/237) were homozygous for the −506 insertion allele (ins/ins). The PAR-1-506 ins/del polymorphism showed a significant association with TTR. Patients with the PAR-1-506 ins/ins genotype had a median TTR of 0.9 years (95% CI: 0.5 to 3.8 years), compared to 1.9 years (95% CI: 1.1-3.1 years) and 4.0 years (95% CI: 2.7-13.2+ years) in patients heterozygous (ins/del) and homozygous (del/del) for the −506 deletion allele, respectively (p=0.003, log-rank test, FIG. 1a).

### EGF+61 A>G (rs4444903) and TTR in Localized Esophageal Adenocarcinoma

Genotyping for EGF+61 A>G was successful in 238 (99%) of 239 cases. In the other 1 patient (1%) genotyping was not successful, because of limited quantity and quality of extracted genomic DNA. Twenty-eight percent (66/238) of patients were homozygous for the EGF+61 A-allele (A/A), 54% (128/238) were heterozygous (A/G), and 18% (44/238) were homozygous for the +61 G-allele (G/G). The EGF+61 A>G polymorphism showed a significant association with TTR. Patients with the EGF+61 A/A genotype had a median TTR of 1.8 years (95% CI: 0.8 to 2.8 years), compared to 3.8 years (95% CI: 2.2 to 13.2+ years) and 3.2 years (95% CI: 1.3 to 6.2+ years) for those heterozygous (A/G) and homozygous (G/G) for the +61 G-allele, respectively (p=0.034, log-rank test, FIG. 1b).

TABLE 4
Multivariable analysis of
EGF +61 A > G and PAR-1 −506 ins/del and TTR

| | | Adjusted RR | Adjusted |
|---|---|---|---|
| | N* | (95% CI)† | P value† |
| EGF +61 A > G (rs4444903) | | | |
| A/A (unfavorable) | 66 | 1 | |
| A/G (favorable) | 128 | 0.58 (0.37, 0.89) | 0.034 |
| G/G (favorable) | 43 | 0.86 (0.49, 1.51) | |
| AG or GG (favorable) | 171 | 0.64 (0.42, 0.96) | |
| PAR-1 −506 ins/del (rs11267092) | | | |
| Del/del (favorable) | 127 | 1 | |
| Ins/del (unfavorable) | 89 | 1.69 (1.14, 2.50) | |
| Ins/ins (unfavorable) | 21 | 1.97 (1.01, 3.87) | 0.015 |
| Ins/del or ins/ins (unfavorable) | 110 | 1.76 (1.21, 2.56) | |
| Combined | | | |
| 2 favorable | 95 | 1 | |
| 1 favorable | 108 | 2.00 (1.30, 3.07) | <0.001 |
| 0 favorable | 34 | 2.59 (1.43, 4.68) | |

*237 patients with complete data of two polymorphisms were included in the model
†Based on the likelihood ratio test within Cox proportional hazards model, adjusted by T1 and N stage, stratified by race, with 2 polymorphisms included.

### Multivariable analysis of PAR-1-506 ins/del (rs11267092) and EGF+61 A>G (rs4444903)

When PAR-1-506 ins/del (adjusted p-value=0.015) and EGF+61 A>G (adjusted p-value=0.034) were analyzed jointly, stratified by race, T-category, and N-category, the two polymorphisms remained significantly associated with TTR (Table 4). In a combined analysis, there was a statistically significant relationship between the two polymorphisms and TTR. Patients harboring two favorable alleles were at lowest risk to develop tumor recurrence (RR=1; reference), compared to patients carrying one (RR=2.0; CI: 1.30-3.07) or no (RR=2.59; CI: 1.43-4.68) favorable alleles, who were at greater risk to develop tumor recurrence (adjusted p-value <0.001; Table 4, FIG. 1c).

### Analysis of Interactions Between of PAR-1-506 ins/del (rs11267092) and EGF+61 A>G (rs4444903) and Race, T-Category, N-Category, and Tumor-Differentiation on Time to Recurrence

It was tested whether the associations between PAR-1-506 ins/del and EGF+61 A>G and time to recurrence differed by sex, race, and type of surgery performed. No significant interactions were found (data not shown).

### Analysis of Other Tested Germline Polymorphisms Involved in the Tumor Angiogenesis Pathway

Statistically significant associations between other tested genes involved in tumor angiogenesis pathway (n=7) and TTR and OS (Table 3) were not observed.

### DISCUSSION

It is herein demonstrated that germline polymorphisms of genes involved in the tumor angiogenesis pathway independently predict tumor recurrence in patients with localized adenocarcinoma of the esophagus. This is the first study to show that angiogenesis-related germline polymorphisms may be important prognostic markers for EA tumor relapse, independent of tumor stage and type of surgery performed.

Numerous studies have demonstrated that the degree of neovascularization in primary cancers directly correlates with tumor growth and—aggressiveness (Folkman (1995) Nat Med 1995; 1(1):27-31). Tumor angiogenesis is driven locally by the release of proangiogenic factors, such as VEGF, bFGF and PDGF. It is well recognized that platelets stimulate endothelial cells in culture and can promote the assembly of capillary-like structures in vitro (Pinedo et al. (1998) Lancet 352(9142):1775-7; Kisucka et al. (2006) Proc Natl Acad Sci USA 103(4):855-60). Accordingly, it has been shown that the proximity to and interaction with the endothelium allow platelets to profoundly influence tumor development. As such, platelet-endothelium interaction modulates the angiogenic balance of the tumor and it has been suggested that platelets may contribute to these angiogenesis dependent processes at least in part through the local release of pro- and antiangiogenic factors (Pinedo et al. (1998) Lancet 352(9142):1775-7; Daly et al. (2003) J Natl Cancer Inst 95(22):1660-73). A recent study by Italiano et al. demonstrated that platelets contain distinct populations of α-granules that can undergo differential release in vitro (Italiano et al. (2008) Blood 111(3):1227-33). Furthermore, the authors demonstrated that at least 2 populations of α-granules containing endogenous angiogenic regulatory proteins are present in platelets and suggest that this subcellular organization is facilitating the differential release of these proteins in response to platelet-endothelial interaction (Italiano et al. (2008) Blood 111(3):1227-33).

Activation of platelets by thrombin is mediated through the cleavage of proteinase-activated receptors (PARs), a receptor family of G-coupled proteins with seven transmembrane proteins (Vu et al. (1991) Cell 64(6):1057-68; Vu et al. (1991) Nature 353(6345):674-7). Four distinct PARs have been indentified, with PAR-1, PAR-3 and PAR-4 acting as receptors for thrombin. Unlike most cellular growth factor receptors, these receptors do not require the traditional ligand-receptor complex formation for activation. In fact, PARs serve as substrates for proteolytic digestion, yielding in irreversible activation of the receptor and in turn promoting transcellular signaling (Vu et al. (1991) Nature 353(6345):674-7; Gerszten et al. (1994) Nature 368(6472):648-51). As such, tumors may modulate PAR activity and its own angiogenic properties through the release of proteases that can either activate or disarm these receptors. In fact, a recent study by Ma et al. revealed that PAR-1 and PAR-4 act in a counter-regulatory manner to influence the local release of VEGF and Endostatin from platelets and as such may profoundly influence tumor-associated angiogenesis (Ma et al. (2005) Proc Natl Acad Sci USA 102(1):216-20). Activation of the Thrombin/PAR-1-receptor axis triggers multiple signaling pathways that result in endothelial cell survival, apoptosis and angiogenesis (Ma et al. (2001) Proc Natl Acad Sci USA 98(11):6470-5; Ward et al. (2001) Cytogenet Cell Genet. 94(3-4):147-54). In addition, over-expression of PAR-1 mRNA and protein was found to correlate with invasive properties of breast carcinoma cell lines and other malignancies (Even-Ram et al. (1998) Nat Med 4(8):909-14; Darmoul et al. (2003) Am J Pathol 162(5):1503-13). DNA sequence variations within the PAR-1 gene may modulate PAR-1 production and/or activity. Among those, a 13-bp insertion/deletion polymorphism (PAR-1-506 ins/del; rs11267092) has been identified, repeating the preceding 5′-CGGCCGCGGGAAG-3′ sequence at position −506 within the promoter region of the PAR-1 gene (Li et al. (1996) J Biol Chem 271(42):26320-8). Interestingly, a potential recognition site (5′-GCGGGAAGC-3′) for activating Ets protein (family of transcription factors) has been identified within the polymorphic 13-bp sequence at position −506 (Li et al. (1996) J Biol Chem 271(42):26320-8). The Ets proteins play a major role in the formation of the transcriptional initiation complex in genes lacking the canonical TATA box sequence, such as the PAR-1 gene promoter (Kamura et al. (1997) J Biol Chem 272(17):11361-8). PAR-1 gene promoter deletion and expression in human endothelial cells showed that the nucleotide sequence −702 to −4, a region encompassing several putative cis-activating sequences, had the highest expression of the reporter gene (Li et al. (1996) J Biol Chem 271(42):26320-8). Because the PAR-1-506 ins/del polymorphism duplicates a putative binding site for Ets-1/Ets-2 and is located within the active part of the promoter, it might influence the gene expression level in basal conditions or in response to different stimuli (Arnaud et al. (2000) Arterioscler Thromb Vasc Biol 20(2):585-92). To date, PAR-1 polymorphisms have not been reported to be causatively linked to time to tumor recurrence or clinical outcome in localized EA patients. In this study, high-expression variants of PAR-1-506 ins/del (ins/ins) were found to be significantly associated with time to tumor recurrence in both univariate and multivariable analysis (Table 2, Table 4, FIG. 1a). These findings demonstrate for the first time that PAR-1-506 ins/del may be an important prognostic factor for EA, supporting the hypothesis that increased angiogenic potential is critical for the development of EA tumor relapse.

The angiogenesis pathway is commonly accepted to have a critical role in survival of esophageal cancer cells. Nevertheless, progression into advanced and recurrent EA has also been associated with activation of other cascades, such as the epidermal growth factor receptor (EGFR) pathway (Vallbohmer & Lenz (2006) Dis Esophagus 19(6):425-32). As such, EGF and its receptor EGFR are proposed to participate in the pathogenesis or maintenance of several human cancers of epithelial origin. In EA, EGFR ligands are frequently elevated and EGFR itself is commonly over-expressed, including up to 92% of esophageal cancers and is associated with tumor progression and poor prognosis (Salomon et al. (1995) Crit. Rev Oncol Hematol 19(3):183-232; Hickey et al. (1994) Cancer 74(6):1693-8; Schneider et al. (2005) J Am Coll Surg 200(3):336-44; Gibault et al. (2005) Br J Cancer 93(1):107-15; Inada et al. (1999) Surg Today 29(6):493-503). Activation of the EGF/EGFR axis triggers multiple signaling pathways that result in endothelial cell proliferation, apoptosis, angiogenesis, and metastasis (Herbst & Shin (2002) Cancer 94(5):1593-611). Moreover, an A to G single nucleotide polymorphism at position +61 in the 5′-untranslated region of the EGF gene (EGF+61 A>G; rs4444903) has been associated with increased risk for numerous malignancies, including EA. Even though conflicting results have been reported about the association between EGF+61 A>G and cancer development and prognosis, several recent studies have reported an adverse effect of the A/A genotype in terms of overall survival and PFS in patients with astrocytoma, colorectal and esophageal adenocarcinoma (Ali-Osman et al. (2006) AACR Meeting Abstracts 2006(1):285-b-; Nagashima et al. (2007) Journal of Clinical Oncology, 2007 ASCO Annual Meeting Proceedings Part I Vol 25, No 18S (June 20 Supplement), 2007: 4129; Graziano et al. (2008) J Clin Oncol 26(9):1427-34; Lanuti et al. (2008) Clin Cancer Res 14(10):3216-22). In fact, Graziano et al. showed that metastatic colorectal cancer (CRC) patients harboring the G-allele, showed to have improved OS, whereas the EGF+61 A/A genotype was associated with poor OS (Graziano et al. (2008) J Clin Oncol 26(9):1427-34). These findings were confirmed by Nagashima et al. (Nagashima et al. (2007) Journal of Clinical Oncology, 2007 ASCO Annual Meeting Proceedings Part I Vol 25, No 18S (June 20 Supplement), 2007: 4129) and Lanuti et al. (Lanuti et al. (2008) Clin Cancer Res 14(10):3216-22), even though later association did not reach statistical significance at the 0.05 level. Indeed, in the present study, the EGF+61 A/A genotype was associated with increased risk of tumor recurrence and not with improved OS. Notably, several independent groups have reported similar findings recently for patients with astrocytoma and CRC, respectively (Ali-Osman et al. (2006) AACR Meeting Abstracts 2006(1):285-b-; Graziano et al. (2008) J Clin Oncol 26(9):1427-34).

Taken together, two independent molecular markers have been identified for tumor recurrence in patients with localized EA. Consistent with the hypothesis, genetic variants of PAR-1-506 ins/del and EGF+61 A>G were independently associated with decreased TTR, indicating a potential role of tumor angiogenesis in the development of EA tumor recurrence. In addition, these findings indicate, that the assessment of the patients' angiogenic potential on the basis of PAR-1 and EGF genotypes might further enhance antiangiogenic treatment not only by the identification of patients at high risk but also by selecting more efficient and tailored treatment strategies.

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

1. A method for identifying a cancer patient as likely or not likely to experience tumor recurrence, comprising determining a genotype of a cell or tissue sample isolated from the patient for at least one polymorphism of the group PAR-1-506 ins/del or EGF+61 A>G, wherein a genotype of at least one of:
(a) (ins/ins or del/ins) for PAR-1-506 ins/del; or
(b) (A/A) for EGF+61 A>G,
identifies the patient as likely to experience tumor recurrence, or a genotype of neither (a) nor (b) identifies the patient as not likely to experience tumor recurrence.

2. The method of claim 1, wherein a genotype of at least one of:
(a) (ins/ins or del/ins) for PAR-1-506 ins/del; or
(b) (A/A) for EGF+61 A>G,
identifies the patient as likely to experience tumor recurrence.

3. The method of claim 1, wherein a genotype of neither (a) nor (b) identifies the patient as not likely to experience tumor recurrence.

4. A method for identifying a cancer patient as more likely or less likely to experience tumor recurrence, comprising determining a genotype of a cell or tissue sample isolated from the patient for at least one polymorphism of the group PAR-1-506 ins/del or EGF+61 A>G, wherein a genotype of at least one of:
(a) (ins/ins or del/ins) for PAR-1-506 ins/del; or
(b) (A/A) for EGF+61 A>G,
identifies the patient as more likely to experience tumor recurrence as compared to a patient having a genotype of neither (a) nor (b) and having the cancer, or a genotype of neither (a) nor (b) identifies the patient as less likely to experience tumor recurrence as compared to a patient having a genotype of at least one of (a) or (b) and having the cancer.

5. The method of claim 4, wherein a genotype of at least one of:
(a) (ins/ins or del/ins) for PAR-1-506 ins/del; or
(b) (A/A) for EGF+61 A>G,
identifies the patient as more likely to experience tumor recurrence as compared to a patient having a genotype of neither (a) nor (b) and having the cancer.

6. The method of claim 4, wherein a genotype of neither (a) nor (b) identifies the patient as less likely to experience tumor recurrence as compared to a patient having a genotype of at least one of (a) or (b) and having the cancer.

7. The method of claim 1, wherein the patient is treated with surgery.

8. The method of claim 7, wherein the cancer patient suffered, before surgery, at least one cancer of the type of the group: esophageal adenocarcinoma, lung cancer, breast cancer, head and neck cancer, ovarian cancer, metastatic or non-metastatic rectal cancer, metastatic or non-metastatic colon cancer, metastatic or non-metastatic colorectal cancer, or non-small cell lung cancer (NSCLC), before being treated with surgery.

9. The method of claim 8, wherein the cancer patient had at least esophageal adenocarcinoma before treated with surgery.

10. The method of claim 1, wherein the sample is at least one of a fixed tissue, a frozen tissue, a biopsy tissue, a resection tissue, a microdissected tissue, or combinations thereof.

11. The method of claim 1, wherein the genotype is determined by a method comprising PCR, PCR-RFLP, sequencing, or microarray.

12. The method of claim 1, wherein the patient is an animal patient.

13. The method of claim 1, wherein the patient is a mammalian, simian, bovine, murine, equine, porcine or ovine patient.

14. The method of claim 1, wherein the patient is a human patient.

15. The method of claim 1, wherein a patient that is more likely to experience tumor recurrence is a patient that has a relatively shorter time to tumor recurrence.

16. The method of claim 1, wherein a patient that is less likely to experience tumor recurrence is a patient that has a relatively longer time to tumor recurrence.

17. A kit for use in determining if a cancer patient treated with surgery is likely to experience tumor recurrence, comprising suitable primers or probes or a microarray for determining at least one polymorphism of the group PAR-1-506 ins/del or EGF+61 A>G, and instructions for use therein.

18. The kit of claim 17, wherein the patient suffered at least one cancer of the type of the group: esophageal adenocarcinoma, lung cancer, breast cancer, head and neck cancer, ovarian cancer, metastatic or non-metastatic rectal cancer, metastatic or non-metastatic colon cancer, metastatic or non-metastatic colorectal cancer, or non-small cell lung cancer (NSCLC) before being treated with surgery.

19. The kit of claim 17, wherein the patient suffered at least esophageal adenocarcinoma before being treated with surgery.

20. The kit of claim 17, wherein the instructions for use comprise instructions for determining the genotype by a method comprising PCR, PCR-RFLP, sequencing, or microarray.

21. A panel of probes or primers or a microarray for determining a genotype of a cell or tissue sample isolated from the patient for polymorphisms of the group PAR-1-506 ins/del and EGF+61 A>G.
